(19) United States
(12) Patent Application Publication
(10) Pub. No.: US 2016/0356723 A1
(43) Pub. Date: Dec. 8, 2016

(54) Light-Up Probes Based On Fluorogens With Aggregation Induced Emission Characteristics For Cellular Imaging And Drug Screening

(71) Applicants: NATIONAL UNIVERSITY OF SINGAPORE, Singapore (SG); THE HONG KONG UNIVERSITY OF SCIENCE AND TECHNOLOGY, Kowloon, Hong Kong (CN)

(72) Inventors: Bin Liu, Singapore (SG); Jinjun Shao, Singapore (SG); Youyong Yuan, Singapore (SG); Jing Liang, Singapore (SG); Benzhong Tang, Kowloon (HK); Zhegang Song, Kowloon (HK); Yilong Chen, Kowloon (HK); Tsz Kin Kwok, Kowloon (HK)

(73) Assignees: NATIONAL UNIVERSITY OF SINGAPORE, Singapore (SG); THE HONG KONG UNIVERSITY OF SCIENCE AND TECHNOLOGY, Kowloon, Hong Kong (HK)

(21) Appl. No.: 15/114,692

(22) PCT Filed: Jan. 27, 2015
(86) PCT No.: PCT/SG2015/000022

§ 371 Date: Jul. 27, 2016

Related U.S. Application Data
(60) Provisional application No. 61/932,007, filed on Jan. 27, 2014.

(51) Int. Cl. G01N 21/77; C07K 7/52; A61K 49/00; A61K 49/14; G01N 33/50; A61K 47/48; C07D 213/38; C07F 9/12; C07K 1/13; C07K 7/06; A61K 33/24

(52) U.S. Cl. CPC G01N 21/77; C07K 7/06; C07K 7/52; A61K 49/0056; A61K 49/146; A61K 33/24; A61K 47/48238; A61K 47/48061; C07D 213/38; C07F 9/12; C07K 1/13; G01N 33/5008; G01N 2333/916; G01N 2333/47; G01N 2021/7786

(57) ABSTRACT

The present invention is drawn toward luminogens and chemical compositions comprising a target recognition motif, a hydrophilic moiety, a linking moiety, and at least one luminogen. Additionally presented are methods of: assessing the conversion of a prodrug into its active form, assessing the therapeutic efficacy of a prodrug, detecting glutathione in a biological sample, detecting alkaline phosphatase in a sample, and conducting fluorescence imaging or magnetic resonance imaging with the use of luminogen-containing compositions.

### RELATED APPLICATION(S)

This application claims the benefit of U.S. Provisional Application No. 61/932,007, filed on Jan. 27, 2014. The entire teachings of the above application are incorporated herein by reference.

### BACKGROUND OF THE INVENTION

Monitoring of intracellular molecules and drug screening can provide valuable insights into the biological conditions of cells and the therapeutic efficiency of drugs. Fluorescence light-up probes based on aggregation-induced emission (AIE) fluorogens and water-soluble peptides have been used for real-time monitoring of cellular proteins. The first generation of specific AIE probe design is limited to peptide based recognition elements with high water solubility. To extend the design principle to include more broad recognition elements (such as, for instance, hydrophobic molecules, small molecules, etc), it is necessary to develop a more general strategy.

### SUMMARY OF THE INVENTION

This invention relates to the development of AIE fluorogens, including those with excited state intramolecular proton transfer (ESIPT) characteristics, AIE light-up probes and their applications in sensing, imaging and drug screening.

More specifically, herein a series of fluorescent light-up probes are described, which generally comprise an AIE fluorogen, a recognition moiety, a targeted ligand and hydrophilic units (e.g. five aspartic acids) to ensure good water-solubility of the probe. Due to the unique nature of the AIE fluorogen, the probes are non-fluorescent in aqueous media but become highly emissive when cleaved by intracellular molecules. The probe enables light-up monitoring of intracellular molecules and drug screening with high signal-to-noise ratio. Based on a similar design principle, replacing the traditional AIE fluorogens with fluorophores showing both AIE and ESIPT characteristics could simplify such design, as the probe is non-fluorescent regardless of the probe water-solubility. As the AIE probe design strategy can be generalized to perform various tasks by simply substituting the recognition moiety to other cleavable linkers in chemical biology, it opens new opportunities to design specific light-up probes for imaging of intracellular molecules and drug screening.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A-1B illustrate an emission spectra and a plot of peak intensities for DMA-HC. FIG. 1A shows the emission spectra of DMA-HC (20 μM) in THF/water mixtures with different fractions of water (f_{w}). FIG. 1B shows the plot of peak intensities versus f_{H}·λₑₓ=430 nm.

FIGS. 2A-2B illustrate an emission spectra and a plot of ratiometric fluorescence intensity for HC-phos. FIG. 2A shows the emission spectra of 40 μM HC-phos in 50 mM Tris-HCl buffer solution (pH 9.2) upon addition of ALP with different activities at 37° C. FIG. 2B shows the plot of ratiometric fluorescence intensity (I₆₄₁/I₅₃₉) versus activities of ALP (incubation time: 45 minutes; λₑₓ=430 nm).

FIG. 3 illustrates bright field and fluorescence images of Hela cells incubated for 25 minutes (excitation wavelength was 460 nm˜490 nm). Images A-B show the bright field and fluorescence images, respectively, of the unstained control. Images C-D show the bright field and fluorescence images, respectively, of cells treated with HC-phos (10 μM). Images E-F show the bright field and fluorescence images, respectively, of cells treated with HC-phos (10 μM) and levamisole (4 mM).

FIG. 4 illustrates HPLC chromatograms showing the reaction of diethyldithiocarbamate (DDTC) with cisplatin and reduced Pt(IV) prodrug. Chromatogram A shows DDTC alone. Chromatogram B shows the reaction of DDTC with cisplatin. Chromatogram C shows the reaction of DDTC with TPS-DEVD-Pt-cRGD. Chromatogram D shows the reaction of DDTC with TPS-DEVD-Pt-cRGD (10 μM) in the presence of 1 mM ascorbic acid for 12 h.

FIGS. 5A-5F illustrate photoluminescence (PL) spectra and/or plots of TPS-CH₂N₃, TPS-DEVD-NH₂ and TPS-DEVD-Pt-cRGD. FIG. 5A shows the PL spectra of TPS-CH₂N₃, TPS-DEVD-NH₂, TPS-DEVD-Pt-cRGD in DMSO/PIPEs (v/v=1/199). The photographic inserts of FIG. 5A were taken under illumination of a UV lamp at 365 nm. FIG. 5B shows the PL spectra of TPS-DEVD-Pt-cRGD upon treatment with ascorbic acid and caspase-3 in the presence and absence of inhibitor 5-[(S)-(+)-2-(methoxymethyl)pyrrolidino]sulfonylisatin (10 μM). FIG. 5C shows the time-dependent fluorescence spectra of TPS-DEVD-Pt-cRGD in the presence of caspase-3 in DMSO/PIPEs buffer (v/v=1/199) after the treatment of ascorbic acid. FIG. 5D shows the PL intensity at 485 nm of ascorbic acid (1 mM) pretreated TPS-DEVD-Pt-cRGD (10 μM) upon addition of caspase-3 (200 μM) from 0 to 120 min. Data of FIG. 5D represent mean values+/−standard deviation, n=3. FIG. 5E shows the PL spectra of ascorbic acid (1 mM) pretreated TPS-DEVD-Pt-cRGD (10 μM) incubated with various amounts of caspase-3 (0, 10, 25, 50, 100 and 200 pM) in DMSO/PIPEs buffer (v/v=1/199) for 60 min. FIG. 5F shows the PL intensity at 485 nm of ascorbic acid (1 mM) pretreated TPS-DEVD-Pt-cRGD (10 μM) upon addition of various amounts of caspase-3 in DMSO/PIPEs buffer (v/v=1/199) for 60 min. Data of FIG. 5F represent mean values+/−standard deviation, n=3.

FIGS. 6A-6B illustrate an (I−I₀)/I₀ plot and a PL spectra of TPS-DEVD-Pt-cRGD. FIG. 6A shows the plot of (I−I₀)/I₀ for TPS-DEVD-Pt-cRGD upon incubation with different proteins for 60 min, where I and I₀ are the PL intensities at protein concentrations of 200 and 0 pM, respectively. FIG. 6B shows the time-dependent PL spectra of TPS-DEVD-Pt-cRGD in apoptotic U87-MG cell lysate (Ap-U87-MG) and normal U87-MG cell lysate (Nor-U87-MG). Data of FIG. 6B represent mean values+/−standard deviation, n=3.

FIG. 7 illustrates CLSM and confocal images as well as fluorescence/nucleus overlay images relating to TPS-DEVD-Pt-cRGD treated cells (all images share the same scale bar of 20 nm). Images A-D show real-time CLSM images displaying the apoptotic progress of TPS-DEVD-Pt-cRGD (5 μM) stained U87-MG cells (nucleuses were living stained with DRAQ5). Images E-F show confocal images of MCF-7 and 293T cells, respectively, upon treatment with TPS-DEVD-Pt-cRGD (5 μM) for 6 h (nucleuses were living stained with DRAQ5). Images G-L are the corresponding fluorescence/nucleus overlay images of images A-F, respectively.

FIG. 8 illustrates CLSM images of U87-MG cells upon treatment with TPS-DEVD-Pt-cRGD (all images share the same scale bar of 20 μm). The three images of part A show the CLSM images of U87-MG cells upon treatment with TPS-DEVD-Pt-cRGD (5 μM), in the absence of inhibitor, and caspase-3 antibody. The three images of part B show the CLSM images of U87-MG cells upon treatment with TPS-DEVD-Pt-cRGD (5 μM), in the presence of inhibitor (5 μM), and caspase-3 antibody.

FIGS. 9A-9B illustrate correlations of cell viability and apoptosis induced PL intensity. FIG. 9A shows the correlations of cell viability (72 h) and apoptosis induced PL intensity (6 h) of U87-MG cells upon treatment with TPS-DEVD-Pt-cRGD at different concentrations. FIG. 9B shows the correlations of cell viability (72 h) and apoptosis induced PL intensity (6 h) of MCF-7 cells upon treatment with TPS-DEVD-Pt-cRGD at different concentrations.

FIGS. 10A-10D illustrate HPLC chromatograms as well as PL spectra and a PL intensity plot. FIG. 10A shows HPLC chromatograms illustrating the reaction of DDTC with cisplatin and reduced Pt(IV) prodrug: (1) DDTC alone, (2) the reaction of DDTC with cisplatin, (3) the reaction of DDTC with PyTPE-Pt-D5-cRGD, (4) the reaction of DDTC with PyTPE-Pt-D5-cRGD (10 μM) in the presence of ascorbic acid (1 mM) for 12 h. FIG. 10B shows the PL spectra of PyTPE-NH2 and PyTPE-Pt-D5-cRGD in DMSO/PBS mixtures (v/v=1/199). The photographic inserts of FIG. 10B were taken under illumination of a UV lamp at 365 nm FIG. 10C shows the time-dependent PL spectra of PyTPE-Pt-D5-cRGD (10 μM) treated with ascorbic acid (1 mM). FIG. 10D shows the plot of PL intensity at 605 nm versus concentrations of PyTPE-Pt-D5-cRGD with the incubation of ascorbic acid (1 mM) in DMSO/PBS (v/v=1/199). Data of FIG. 10D represent mean values+/−standard deviation, n=3.

FIG. 11 illustrates confocal microscopy images of MDA-MB-231 and MCF-7 cells after incubation (all images share the same scale bar of 20 μm). Image A shows the confocal image of MDA-MB-231 cells after incubation with PyTPE-Pt-D5-cRGD (the nuclei were stained with Hoechst 33342). Image B shows the confocal image of MDA-MB-231 cells after incubation with PyTPE-Pt-D5 (the nuclei were stained with Hoechst 33342). Image C shows the confocal image of MDA-MB-231 cells after incubation with PyTPE-C6-D5-cRGD (the nuclei were stained with Hoechst 33342). Image D shows the confocal image of MCF-7 cells after incubation with PyTPE-Pt-D5-cRGD (the nuclei were stained with Hoechst 33342). Image E shows the confocal image of MCF-7 cells after incubation with PyTPE-Pt-D5 (the nuclei were stained with Hoechst 33342). Image F shows the confocal image of MCF-7 cells after incubation with PyTPE-C6-D5-cRGD (the nuclei were stained with Hoechst 33342).

FIGS. 12A-12B illustrate the cell viability of MDA-MB-231 and MCF-7 cells upon treatment. FIG. 12A shows the cell viability of MDA-MB-231 cells upon treatment with PyTPE-Pt-D5-cRGD, PyTPE-Pt-D5 and PyTPE-C6-D5-cRGD at different concentrations for 72 h. FIG. 12B shows the cell viability of MCF-7 cells upon treatment with PyTPE-Pt-D5-cRGD, PyTPE-Pt-D5 and PyTPE-C6-D5-cRGD at different concentrations for 72 h.

FIGS. 13A-13D illustrate PL spectra as well as a plot of PL intensity. FIG. 13A shows the PL spectra of TPE-CH₂NH₂ and TPE-SS-D5-cRGD in DMSO/PBS (v/v=1/199). The photographic inserts of FIG. 13A were taken under illumination of a UV lamp. FIG. 13B shows the time-dependent PL spectra of TPE-SS-D5-cRGD treated with GSH. FIG. 13C shows the PL spectra of TPE-SS-D5-cRGD (1.0 mM) in the presence of different concentrations of GSH. FIG. 13D shows the plot of PL intensity at 470 nm versus concentrations of GSH (mean+/−standard deviation, n=3).

FIG. 14 illustrates confocal microscopy images of U87-MG and MCF-7 cells after incubation (all images share the same scale bar of 20 μm). Image A shows the confocal image of U87-MG cells after incubation with TPE-SS-D5-cRGD (nuclei were stained with propidium iodide). Image B shows the confocal image of U87-MG cells after incubation with TPE-SS-D5 (nuclei were stained with propidium iodide). Image C shows the confocal image of U87-MG cells after incubation with TPE-CC-D5 (nuclei were stained with propidium iodide). Image D shows the confocal image of MCF-7 cells after incubation with TPE-SS-D5-cRGD (nuclei were stained with propidium iodide). Image E shows the confocal image of MCF-7 cells after incubation with TPE-SS-D5 (nuclei were stained with propidium iodide). Image F shows the confocal image of MCF-7 cells after incubation with TPE-CC-D5 (nuclei were stained with propidium iodide).

### DETAILED DESCRIPTION OF THE INVENTION

The present invention is directed to luminogens (AIE fluorogens and AIE-ESIPT fluorogens being subclasses thereof) and chemical compositions (e.g. light-up probes) comprising a target recognition motif, a hydrophilic moiety, a linking moiety, and at least one luminogen. The present invention is also directed to methods of assessing the conversion of a prodrug into its active form, assessing the therapeutic efficacy of a prodrug, detecting glutathione in a biological sample, detecting alkaline phosphatase in a sample, and conducting fluorescence imaging or magnetic resonance imaging with the use of said compositions comprising the luminogens.

### DEFINITIONS

All definitions of substituents set forth below are further applicable to the use of the term in conjunction with another substituent.

“Alkyl” means a saturated aliphatic branched or straight-chain monovalent hydrocarbon radical, typically C₁-C₁₀, preferably C₁-C₆. “(C₁-C₆) alkyl” means a radical having from 1-6 carbon atoms in a linear or branched arrangement. “(C₁-C₆)alkyl” includes methyl, ethyl, propyl, butyl, tert-butyl, pentyl and hexyl.

“Alkylene” means a saturated aliphatic straight-chain divalent hydrocarbon radical. Thus, “(C₁-C₆)alkylene” means a divalent saturated aliphatic radical having from 1-6 carbon atoms in a linear arrangement. “(C₁-C₆)alkylene” includes methylene, ethylene, propylene, butylene, pentylene and hexylene.

“Cycloalkyl” means saturated aliphatic cyclic hydrocarbon ring. Thus, “C₃-C₈ cycloalkyl” means (3-8 membered) saturated aliphatic cyclic hydrocarbon ring. C₃-C₃ cycloalkyl includes, but is not limited to cyclopropyl, cyclobutyl, cyclopentyl, cyclohexyl, cycloheptyl, and cyclooctyl. Preferably, cycloalkyl is C₃-C₆ cycloalkyl.

The term “alkoxy” means —O-alkyl; “hydroxyalkyl” means alkyl substituted with hydroxy; “aralkyl” means alkyl substituted with an aryl group; “alkoxyalkyl” mean alkyl substituted with an alkoxy group; “alkylamine” means amine substituted with an alkyl group; “cycloalkylalkyl” means alkyl substituted with cycloalkyl; “dialkylamine” means amine substituted with two alkyl groups; “alkylcarbonyl” means —C(O)-A*, wherein A* is alkyl; “alkoxycarbonyl” means C(O) OA*, wherein A* is alkyl; and where alkyl is as defined above. Alkoxy is preferably O(C₁-C₆)alkyl and includes methoxy, ethoxy, propoxy, butoxy, pentoxy and hexoxy.

“Cycloalkoxy” means a —O-cycloalkyl, wherein the cycloalkyl is as defined above. Exemplary (C₃-C₇)cycloalkyloxy groups include cyclopropoxy, cyclobutoxy, cyclopentoxy, cyclohexoxy and cycloheptoxy.

The term “aryl” used alone or as part of a larger moiety as in “arylalkyl”, “arylalkoxy”, “aryloxy”, or “aryloxyalkyl”, means carbocyclic aromatic rings. The term “carbocyclic aromatic group” may be used interchangeably with the terms “aryl”, “aryl ring” “carbocyclic aromatic ring”, “aryl group” and “carbocyclic aromatic group”. An aryl group typically has 6-16 ring atoms. A “substituted aryl group” is substituted at any one or more substitutable ring atom. The term “C₆-C₁₆ aryl” as used herein means a monocyclic, bicyclic or tricyclic carbocyclic ring system containing from 6 to 16 carbon atoms and includes phenyl (Ph), naphthyl, anthracenyl, 1,2-dihydronaphthyl, 1,2,3,4-tetrahydronaphthyl, fluorenyl, indanyl, indenyl and the like. In particular embodiments, the aryl group is (C₆-C₁₀)aryl. The (C₆-C₁₀)aryl(C₁-C₆)alkyl group connects to the rest of the molecule through the (C₁-C₆)alkyl portion of the (C₆-C₁₀)aryl(C₁-C₆)alkyl group.

“Hetero” refers to the replacement of at least one carbon atom member in a ring system with at least one heteroatom selected from N, S, and O. The heteroatom can optionally carry a charge. When N is the heteroatom of a ring system, it may be additionally substituted by one or more substituents including H, OH, O-, alkyl, aryl, heterocyclyl, cycloalkyl or alkenylene, wherein any of the alkyl, aryl, heterocyclyl, cycloalkyl or alkenylene may be optionally and independently substituted by one or more substituents selected from halo, cyano, nitro, hydroxyl, phosphate (PO₄³⁻) or a sulfonate (SO³⁻).

“Heterocycle” means a saturated or partially unsaturated (3-7 membered) monocyclic heterocyclic ring containing one nitrogen atom and optionally 1 additional heteroatom independently selected from N, O or S. When one heteroatom is S, it can be optionally mono- or di-oxygenated (—S(O)— or S(O)₂). Examples of monocyclic heterocycle include, but not limited to, azetidine, pyrrolidine, piperidine, piperazine, hexahydropyrimidine, tetrahydrofuran, tetrahydropyran, morpholine, thiomorpholine, thiomorpholine 1,1-dioxide, tetrahydro-2H-1,2-thiazine, tetrahydro-2H-1,2-thiazine 1,1-dioxide, isothiazolidine, or isothiazolidine 1,1-dioxide. The heterocycle can be optionally fused to a carbocyclic ring, as in, for example, indole.

The term “heteroaryl”, “heteroaromatic”, “heteroaryl ring”, “heteroaryl group” and “heteroaromatic group”, used alone or as part of a larger moiety as in “heteroarylalkyl” or “heteroarylalkoxy”, refers to aromatic ring groups having five to fourteen total ring atoms selected from carbon and at least one (typically 1-4, more typically 1 or 2) heteroatoms (e.g., oxygen, nitrogen or sulfur). They include monocyclic rings and polycyclic rings in which a monocyclic heteroaromatic ring is fused to one or more other carbocyclic aromatic or heteroaromatic rings. The term “5-14 membered heteroaryl” as used herein means a monocyclic, bicyclic or tricyclic ring system containing one or two aromatic rings and from 5 to 14 total atoms of which, unless otherwise specified, one, two, three, four or five are heteroatoms independently selected from N, NH, N(C₁₋₆alkyl), 0 and S. (C₃-C₁₀)heteroaryl includes furyl, thiophenyl, pyridinyl, pyrrolyl, imidazolyl, and in preferred embodiments of the invention, heteroaryl is (C₃-C₁₀)heteroaryl.

“Halogen” and “halo” are interchangeably used herein and each refers to fluorine, chlorine, bromine, or iodine.

“Cyano” means —C≡N.

“Nitro” means —NO₂.

As used herein, an amino group may be a primary (NH₂), secondary (NHRₓ), or tertiary (NRₓR_{y}), wherein Rₓ and R_{y} may be any alkyl, aryl, heterocyclyl, cycloalkyl or alkenylene, each optionally and independently substituted with one or more substituents described above. The Rₓ and R_{y} substituents may be taken together to form a “ring”, wherein the “ring”, as used herein, is cyclic amino groups such as piperidine and pyrrolidine, and may include heteroatoms such as in morpholine.

The terms “haloalkyl”, “halocycloalkyl” and “haloalkoxy” mean alkyl, cycloalkyl, or alkoxy, as the case may be, substituted with one or more halogen atoms. The term “halogen” means F, Cl, Br or I.

The term “acyl group” means —C(O)A*, wherein A* is an optionally substituted alkyl group or aryl group (e.g., optionally substituted phenyl).

An “alkylene group” is represented by —[CH₂]_{z}, wherein z is a positive integer, preferably from one to eight, more preferably from one to four.

An “alkenylene group” is an alkylene in which at least a pair of adjacent methylenes are replaced with —CH═CH.

The term benzyl (Bn) refers to —CH₂Ph.

The term “Alkenyl” means a straight or branched hydrocarbon radical including at least one double bond. The (C₆-C₁₀)aryl(C₂-C₆)alkenyl group connects to the remainder of the molecule through the (C₂-C₆)alkenyl portion of (C₆-C₁₀)aryl(C₂-C₆)alkenyl.

Pharmaceutically acceptable salts of the compounds of the present invention are also included. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the compounds of the present invention containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

The term “luminogen”, as used herein, refers to a molecule that exhibits light-emission. If the light that is emitted is fluorescent light, the luminogen is alternately referred to as a fluorogen.

“Aggregation-induced emission” refers to a property in which a luminogen, when dispersed, for example in organic solvent, emits little or no light. Upon aggregation of luminogen molecules, however, for example in the solid state or in water due to the hydrophobicity of the luminogen, light emission from the luminogen is significantly enhanced.

A “target recognition motif” as used herein, is a chemical moiety having an affinity for a biological target such as a protein, a peptide, or a receptor in the cell membrane. A target recognition motif can comprise a peptide, a protein, an oligonucleotide, or an organic functional group having an affinity for a specific target structure.

A “linking moiety” as used herein, is a chemical moiety that links two or more groups through one covalent bond or through a series of covalent bonds. Example linking moieties include disulfide groups, amino groups, 2-nitrobenzyl derivatives, sulfones, hydrazones, vicinal diols, or simply one or more covalent bonds. Further examples of linking moieties can be found in Table 1 of Bioorg. Med. Chem., 2012, 20, 571-582.

Hydrophilic moieties for use in the compositions of the present invention can include water soluble polymers or alkyl chains functionalized by charged side groups. Examples of water soluble polymers for use with the present invention include polyethylene glycol or polyethylenimine. Charged side groups that may be used with the present invention include, for example SO₃³⁻ or PO₄³⁻.

As used herein, “spectroscopy” encompasses any method by which matter reacts with radiated energy. This includes, but is in no way limited to, microscopy, fluorescence microscopy, UV/Vis spectrometry, and flow cytometry. A “microplate reader” as used herein, means a laboratory instrument that measures, for example, fluorescence, absorbance and luminescence of samples contained in a microplate.

A “prodrug” as used herein, is a therapeutic compound that is typically administered to a subject in its inactive form and is converted to its active form in the body of the subject. For example, a prodrug may include a platinum (IV) [Pt (IV)] complex that is converted to an active platinum (II) [Pt (II)] complex. In certain embodiments, such a conversion occurs via reduction with a chemical reagent. In certain other embodiments, such a conversion occurs via metabolic processes.

Tetraphenylethylene, or TPE, is:

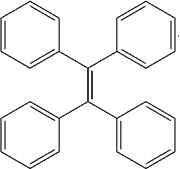 [SMILES: c1ccc(C(=C(c2ccccc2)c2ccccc2)c2ccccc2)cc1]

Tetraphenylsilole, or TPS, is:

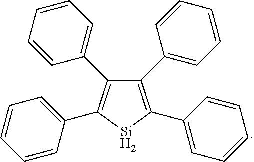

“Live target cells” as used herein, are live cells that are the target of a treatment or therapeutic regimen. In some embodiments, live target cells can be cancer cells that are the therapeutic target of a prodrug.

A description of various aspects of the invention follows.

### Luminogens

In this first aspect of the invention are described luminogens. A luminogen is an atom, or groups of atoms, that luminesces. Similar to a luminogen, a fluorogen is an atom, or groups of atoms, that fluoresces. Luminescence is a process of emitting light. Types of luminescence include bioluminescence, chemiluminescence, electroluminescence, electrochemiluminescence, photoluminescence, and others. A type of photoluminescence is known as fluorescence. Thus, fluorogens are a subset of luminogens.

### AIE Fluorogens

Within the family of fluorogens can be found aggregation-induced emission (AIE) fluorogens. One embodiment of this aspect of the present invention is directed to an AIE fluorogen having the structure of formula:

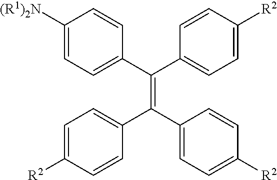

or a pharmaceutically acceptable salt thereof, wherein:

R¹ is selected from H, (C₁-C₆)alkyl, (C₃-C₆)cycloalkyl, (C₆-C₁₀)aryl, (C₃-C₁₀)heteroaryl, or (C₂-C₆)alkenyl;

R² is independently selected from H, NHR³, N(R³)₂, (C₁-C₆)alkyl, (C₃-C₆)cycloalkyl, (C₆-C₁₀)aryl, (C₃-C₁₀)heteroaryl, —O(C₁-C₆)alkyl, (C₂-C₆)alkenyl, CH═CH((C₃-C₁₀)heteroaryl), or CH═CH((C₆-C₁₀)aryl); and

R³ is selected from H, (C₁-C₆)alkyl or (C₃-C₆)cycloalkyl.

The AIE fluorogen is also optionally and independently substituted with one or more substituents selected from: (C₃-C₁₀)heteroaryl,

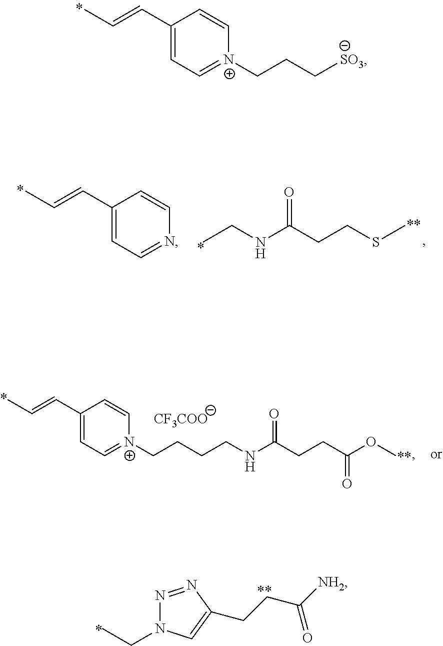

wherein * indicates the point of attachment to the luminogen residue and ** indicates the point of attachment to either the prodrug, the target recognition motif or the hydrophilic peptide.

In another embodiment, the AIE fluorogen has the structure of formula:

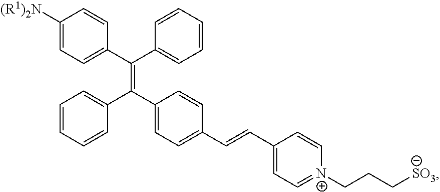

wherein R¹ is (C₁-C₆)alkyl. In a preferred embodiment, R¹ is C₂H₅ or C₆H₁₃.

In yet another embodiment, the AIE fluorogen has the structure of formula:

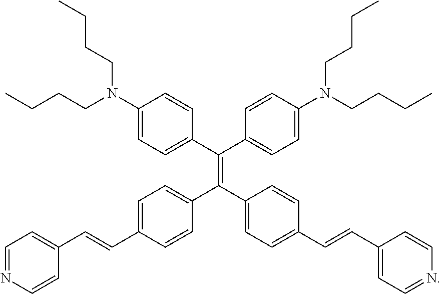 [SMILES: CCCCN(CCCC)c1ccc(C(=C(c2ccc(/C=C/c3ccncc3)cc2)c2ccc(/C=C/c3ccncc3)cc2)c2ccc(N(CCCC)CCCC)cc2)cc1]

The following schemes more specifically illustrate the design and synthesis of several exemplary AIE fluorogens.

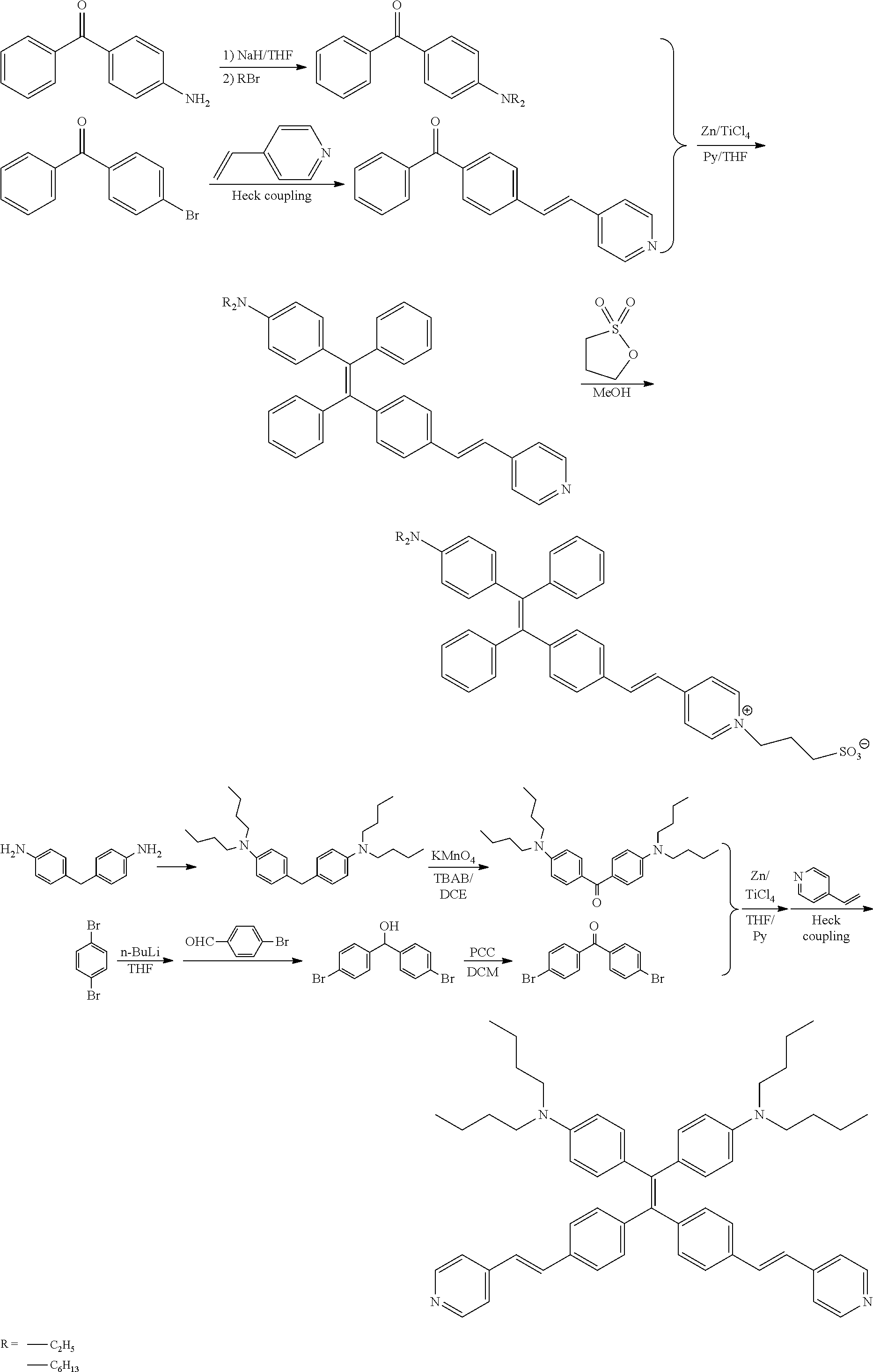

More detailed synthetic routes for exemplary AIE fluorogens can be found in the Exemplification section of this application.

### AIE-ESIPT Fluorogens

Within the family of AIE fluorogens can be found those with excited state intramolecular proton transfer (ESIPT) characteristics (AIE-ESIPT fluorogens).

One embodiment of this aspect of the present invention is drawn toward a fluorogen having the structure of formula:

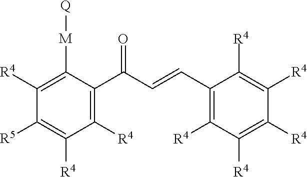

or a pharmaceutically acceptable salt thereof, wherein:

M is selected from S, O or NH;

Q is selected from P(═O)(OH)₂ or C(O)O(C₁-C₆)alkyl, wherein C(O)O(C₁-C₆)alkyl is optionally functionalized by one or more substituents selected from SH, OH, NH₂, or (C₆-C₁₀)aryl optionally substituted with one or more substituents selected from OH, SH, or NH₂;

R⁴ is selected from NHR⁶, N(R⁶)₂, (C₁-C₆)alkyl, (C₃-C₆)cycloalkyl, (C₆-C₁₀)aryl, (C₃-C₁₀)heteroaryl, —O(C₁-C₆)alkyl, —O(C₃-C₆)cycloalkyl or (C₂-C₆)alkenyl;

R⁵ is (C₀-C₆)alkyl, optionally functionalized by a linking moiety; and

R⁶ is selected from H, (C₁-C₆)alkyl or (C₃-C₆)cycloalkyl.

In a preferred embodiment, the linking moiety, if present, is covalently attached to a target recognition motif. The target recognition motif preferably has an affinity for a cell membrane receptor. More preferably, the target recognition motif has a cyclic (Arg-Gly-Asp) (cRGD) residue having an affinity for integrin αᵥβ₃.

In another embodiment, the AIE-ESIPT fluorogen has the structure of formula:

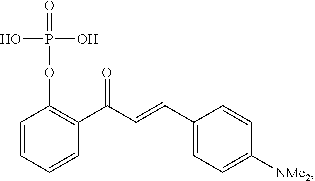 [SMILES: CN(C)c1ccc(/C=C/C(=O)c2ccccc2OP(=O)(O)O)cc1]

known as Phos-HC.

The following scheme more specifically illustrates the design and synthesis of an exemplary AIE-ESIPT fluorogen.

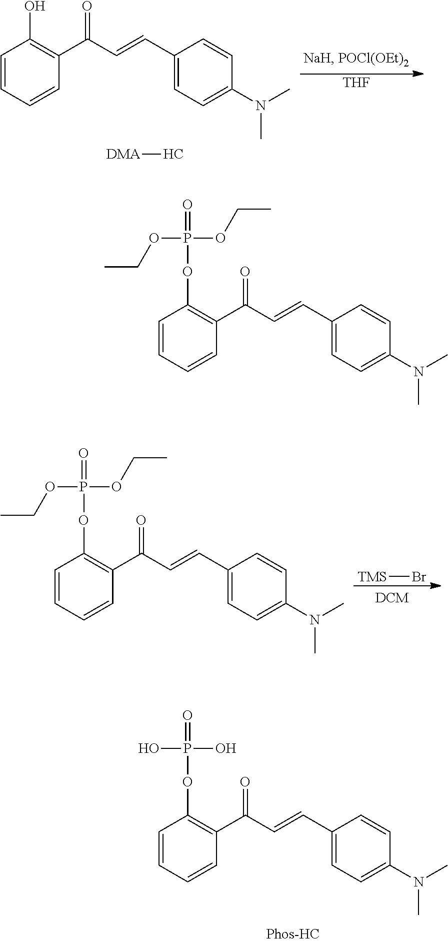

Use of this exemplary AIE fluorogen in the detection of alkaline phosphatase in a sample can be found in the Exemplification section of this application.

Both AIE fluorogens and AIE-ESIPT fluorogens can be used in the creation of light-up probes for conducting fluorescence imaging and magnetic resonance imaging as well as for assessing the conversion of a prodrug into its active form, assessing the therapeutic efficacy of a prodrug, detecting glutathione in a biological sample, and detecting alkaline phosphatase in a sample.

### Light-Up Probes

Targeted drug delivery to tumor cells with minimized side effects and real-time in-situ monitoring of drug efficacy is highly desirable for personalized medicine. More specifically, it is highly desirable if one could design and develop a system that can simultaneously deliver drugs and non-invasively evaluate the therapeutic responses in-situ. The most promising solution to this issue is to incorporate an apoptosis sensor into the system.

The inventors of the present invention have developed a strategy for real-time monitoring of cell apoptosis in-vitro and in-vivo based on probes containing fluorogens (light-up probes) with AIE characteristics. The composition of the light-up probes will be discussed first, followed by a description of the applications of the light-up probes.

In the light-up probe aspect of the present invention, the light-up probe is a chemical composition comprising at least one luminogen, a hydrophilic moiety, a linking moiety, and a target recognition motif, wherein the luminogen exhibits aggregation-induced emission properties, and further wherein the target recognition motif, the hydrophilic moiety, the linking moiety, and the at least one luminogen are linked by covalent linkages in a linear array.

In one embodiment, the linking moiety is a prodrug, such as for example a platinum (IV) complex (“Pt”).

In another embodiment, the linking moiety is a cleavable linking group. Preferably, a cleavable linking moiety is a disulfide (“SS”). Alternatively, the cleavable linking group can be a hydrazone bond that can be cleaved in acidic conditions or an aminoacrylate (AA) linker that can be cleaved by reactive oxygen species.

In one embodiment, the hydrophilic moiety comprises a hydrophilic peptide, a self-assembling peptide, an oligonucleotide, a water-soluble polymer, or an alkyl chain functionalized by charged side groups. Preferably, the alkyl chain has greater than five carbon atoms and the charged side groups can be, for example, an amine group, a carboxyl group or a guanidinium group.

In one embodiment, the hydrophilic moiety is a hydrophilic peptide. For example, the hydrophilic peptide can comprise an amino acid residue sequence comprising at least one of Lys, Asp, Arg, His or Glu. Preferred hydrophilic peptides can be Asp-Asp-Asp-Asp-Asp (SEQ ID NO:1) (“D5”) or Asp-Glu-Val-Asp (SEQ ID NO:2) (“DEVD”).

Preferred self-assembling peptides are (Ala-Glu-Ala-Glu-Ala-Lys-Ala-Lys)₂ (SEQ ID NO:3) or Phe-Phe.

The target recognition motif preferably has an affinity for a cell membrane receptor. Preferably, the target recognition motif has a cyclic (Arg-Gly-Asp) residue (“cRGD”) having an affinity for integrin αᵥβ₃. Alternatively, the target recognition motif can be a lysosomal protein transmembrane 4 beta.

The luminogens of the light-up probes of the present invention comprise at least those fluorogens described herein, as well as tetraphenylsilole (“TPS”), tetraphenylethene pyridinium (“PyTPE”) and tetraphenylethylene (“TPE”).

The components of the light-up probe composition are covalently linked in a linear array. The determination of components and the order of linkage can be varied and are selected based on the desired application of the light-up probe. The following Table illustrates exemplary component selections as well as exemplary orders of linkage of said components (read from left to right).

| | 1st Component | | | 2nd & 3rd Components | | | | | | | | 4th Component |
|---|---|---|---|---|---|---|---|---|---|---|---|---|
| | luminogen | | | hydrophilic moiety-linking moiety or linking moiety-hydrophilic moiety | | | | | | | | target recognition motif |
| row | TPS- | PyTPE- | TPE- | DEVD-Pt | DEVD-SS | D5-Pt | D5-SS | Pt-DEVD | SS-DEVD | Pt-D5 | SS-D5 | -cRGD |
| A | TPS- | | | DEVD-Pt | | | | | | | | -cRGD |
| B | TPS- | | | | DEVD-SS | | | | | | | -cRGD |
| C | TPS- | | | | | D5-Pt | | | | | | -cRGD |
| D | TPS- | | | | | | D5-SS | | | | | -cRGD |
| E | TPS- | | | | | | | Pt-DEVD | | | | -cRGD |
| F | TPS- | | | | | | | | SS-DEVD | | | -cRGD |
| G | TPS- | | | | | | | | | Pt-D5 | | -cRGD |
| H | TPS- | | | | | | | | | | SS-D5 | -cRGD |
| I | | PyTPE- | | DEVD-Pt | | | | | | | | -cRGD |
| J | | PyTPE- | | | DEVD-SS | | | | | | | -cRGD |
| K | | PyTPE- | | | | D5-Pt | | | | | | -cRGD |
| L | | PyTPE- | | | | | D5-SS | | | | | -cRGD |
| M | | PyTPE- | | | | | | Pt-DEVD | | | | -cRGD |
| N | | PyTPE- | | | | | | | SS-DEVD | | | -cRGD |
| O | | PyTPE- | | | | | | | | Pt-D5 | | -cRGD |
| P | | PyTPE- | | | | | | | | | SS-D5 | -cRGD |
| Q | | | TPE- | DEVD-Pt | | | | | | | | -cRGD |
| R | | | TPE- | | DEVD-SS | | | | | | | -cRGD |
| S | | | TPE- | | | D5-Pt | | | | | | -cRGD |
| T | | | TPE- | | | | D5-SS | | | | | -cRGD |
| U | | | TPE- | | | | | Pt-DEVD | | | | -cRGD |
| V | | | TPE- | | | | | | SS-DEVD | | | -cRGD |
| W | | | TPE- | | | | | | | Pt-D5 | | -cRGD |
| X | | | TPE- | | | | | | | | SS-D5 | -cRGD |

This table is for illustration purposes only and does not reflect all possible component selections nor does it reflect all possible orders of linkage. For instance, when AIE-ESIPT fluorogens are used as the luminogen, the hydrophilic moiety can be removed, and when two luminogens are present, the second luminogen can be inserted after the third component (hydrophilic moiety or linking moiety) but before the fourth component (target recognition motif).

In another embodiment of the light-up probe aspect of the present invention, there are specific AIE light-up probes with dual imaging functionality. An example of such a probe is TPE-DEVD-DOTA/Gd (“DDT-Gd”).

Detailed synthetic routes as well as testing parameters and results for several exemplary probes (e.g. the probes of Table rows A, O and X, and the DDT-Gd probe) can be found in the Exemplification section of this application.

The light-up probes of the present invention can be used generally for conducting fluorescence imaging and magnetic resonance imaging and specifically for assessing the conversion of a prodrug into its active form, assessing the therapeutic efficacy of a prodrug, detecting glutathione in a biological sample, and detecting alkaline phosphatase in a sample. A more detailed description of the applications aspect of the invention follows.

### Applications

While the light-up probes of the present invention can be used for conducting fluorescence imaging and magnetic resonance imaging generally, the following discussion will focus on uses for non-invasive early evaluation of therapeutic responses in-situ, selective and real-time monitoring of drug activation in-situ, targeted intracellular thiol imaging, and alkaline phosphatase (ALP) detection.

### Non-Invasive Early Evaluation of Therapeutic Responses In-Situ

The inventors of the present invention have developed a strategy for real-time monitoring of cell apoptosis in-vitro and in-vivo based on light-up probes of the present invention that contain fluorogens with AIE (or AIE-ESIPT) characteristics.

An embodiment of this aspect of the present invention thus pertains to a method for assessing the therapeutic efficacy of a prodrug, comprising:

a) incubating a biological sample comprising live target cells with a chemical composition of the invention under conditions sufficient to convert the prodrug into its active form, to form an incubated mixture; and

b) analyzing the incubated mixture of step a) by fluorescence spectroscopy,

wherein an increase in fluorescence intensity as compared to the fluorescence intensity of the chemical composition not in the presence of the biological sample is indicative of the efficacy of the active drug.

With specific reference to the light-up probe TPS-DEVD-Pt-cRGD (see row A of Table), the method of the present invention for assessing the therapeutic efficacy of a prodrug will be explained and described in more detail as follows.

A targetable theranostic Pt(IV) prodrug was developed with a special focus on monitoring drug induced apoptosis in-situ. The theranostic system comprises a chemotherapeutic Pt(IV) prodrug which can be reduced to active Pt(II) intracellularly, an apoptosis sensor (TPS-DEVD) based on tetraphenylsilole (TPS) with AIE characteristic and a cyclic (RGD) peptide as a targeting ligand (Scheme 1). The prodrug can accumulate preferentially in cancer cells with overexpressed αᵥβ₃ integrin and release the active drug Pt(II) and apoptosis sensor TPS-DEVD upon the intracellular reduction of Pt(IV) prodrug. The released Pt(II) can induce cell apoptosis and activate caspase-3 to cleave the DEVD peptide in TPS-DEVD and trigger fluorescence. The fluorescence turn-on response can be utilized in the theranostic system for real-time and noninvasive imaging of therapeutic responses of a specific anticancer drug. The cancer cells of the theranostic system include, for example, U87-MG, MDA-MB-231 and HT29. Contemplated anticancer drugs include, for instance, doxorubicin and paclitaxel.

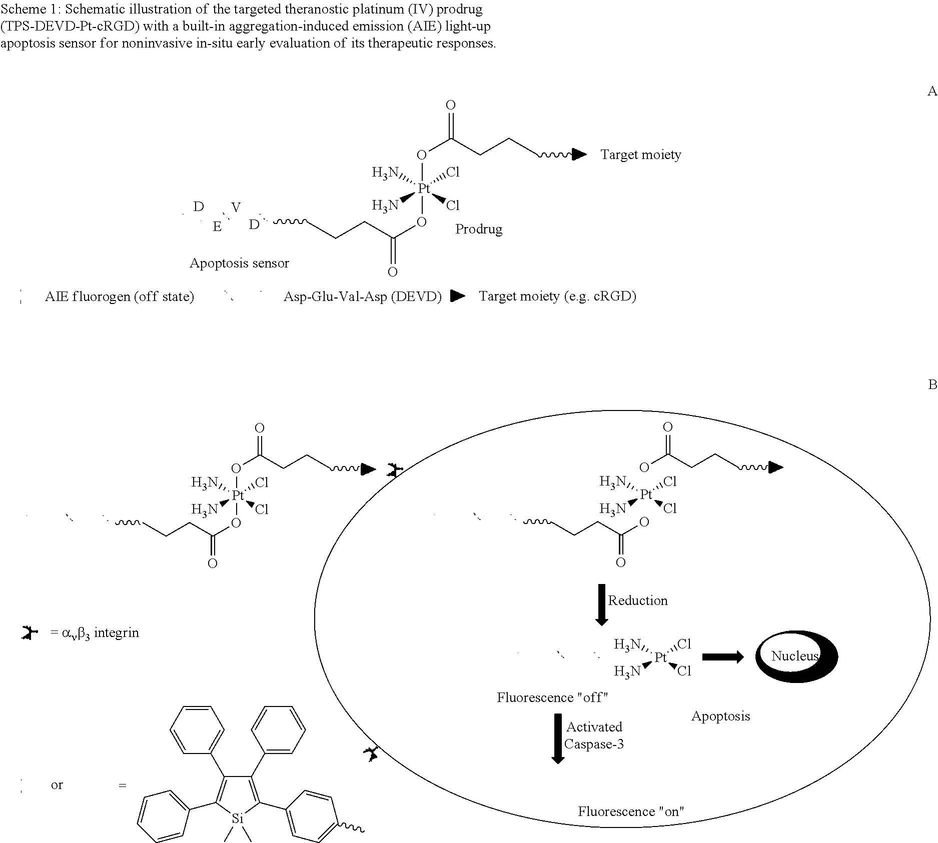

### Selective and Real-Time Monitoring of Drug Activation In-Situ

The inventors of the present invention have developed a simple strategy for in situ monitoring of drug activation utilizing the light-up probes of the present invention that contain fluorogens with AIE (or AIE-ESIPT) characteristics.

An embodiment of this aspect of the present invention thus pertains to a method for assessing the conversion of a prodrug into its active form, the method comprising:

a) incubating a biological sample with the above-noted chemical composition under conditions sufficient to form an incubated mixture; and

b) analyzing the fluorescence of the incubated mixture of step a) using a microplate reader,

wherein an increase in fluorescence intensity as compared to the fluorescence intensity of the above-noted chemical composition not in the presence of the biological sample is indicative of the conversion of the prodrug into its active form.

Preferably this method is conducted in a live cell.

With specific reference to the light-up probe PyTPE-Pt-D5-cRGD (see row O of Table), the method of the present invention for assessing the conversion of a prodrug into its active form will be explained and described in more detail below.

The design and synthesis of a targeted theranostic platinum(IV) prodrug delivery system was developed. This system was based on an AIE luminogen for in situ monitoring of the platinum(IV) prodrug activation. The theranostic system, comprises a chemotherapeutic prodrug Pt(IV) that can be reduced to active Pt(II) inside the cells, a tetraphenylethene pyridinium (PyTPE) unit with AIE characteristics, a short hydrophilic peptide with five aspartic acid (D5) units to ensure its water solubility and a cyclic (RGD) peptide (cRGD) as a targeting ligand (Scheme 2). The prodrug can accumulate preferentially in cancer cells that overexpress αᵥβ₃ integrin and can be utilized as an excellent guiding molecule to tumor cells, for example, U87-MG, MDA-MB-231 and HT29 cells. In aqueous media, the AIE moiety is non-fluorescent due to the high hydrophilicity of the D5-cRGD, but its emission is enhanced significantly after the reduction of the Pt(IV) complex, which releases the two axials. The fluorescent enhancement (“turn-on”) is attributed to the restriction of intramolecular rotations of the PyTPE phenyl rings in the cleaved residues, which populates the radiative decay channels. The prodrug design of the invention offers good opportunity for efficient targeted platinum drug delivery and real-time monitoring of the release and distribution of the drug with a high signal-to-noise ratio.

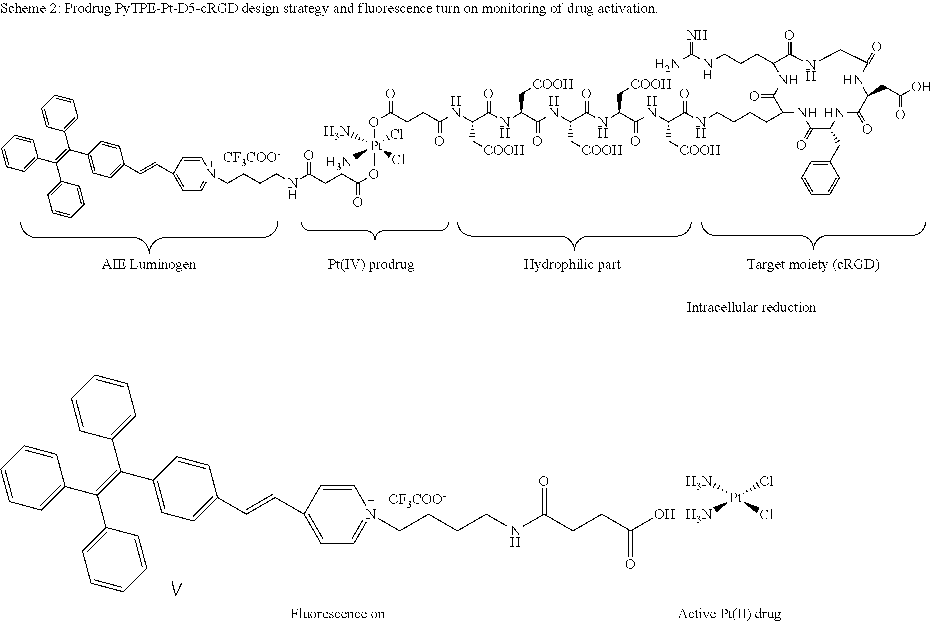

### Targeted Intracellular Thiol Imaging

The inventors of the present invention have developed a strategy for cell specific intracellular thiol (e.g. glutathione) imaging utilizing the light-up probes of the present invention that contain fluorogens with AIE (or AIE-ESIPT) characteristics.

An embodiment of this aspect of the present invention thus pertains to a method of detecting glutathione in a biological sample, the method comprising:

a) incubating a biological sample thought to contain glutathione with the above-noted chemical composition under conditions sufficient to form an incubated mixture; and

b) analyzing the incubated mixture of step a) by fluorescence spectroscopy,

wherein an increase in fluorescence intensity as compared to the fluorescence intensity of the above-noted chemical composition not in the presence of the biological sample is indicative of the presence of glutathione.

In the method of detecting glutathione, the fluorescence intensity of the incubated mixture preferably increases with increased concentration of glutathione.

With specific reference to the light-up probe TPE-SS-D5-cRGD (see row X of Table), the method of the present invention for detecting glutathione will be explained and described in more detail below.

An integrin αᵥδ₃ targeted light-up probe was designed for cell specific intracellular thiol imaging. The probe comprises a targeted cyclic RGD (cRGD) peptide, a highly water soluble peptide with five aspartic acids (Asp, D5), a TPE fluorogen and a thiol-specific cleavable disulfide linker. cRGD exhibits high binding affinity to αᵥβ₃ integrin which is a unique molecular biomarker for early detection and treatment of rapidly growing solid tumors comprising, for example, U87-MG, MDA-MB-231 and HT29 cancer cells. The probe is highly water soluble and is almost non-fluorescent in aqueous media. The cleavage of the disulfide group by thiols leads to enhanced fluorescence signal output (Scheme 3). This probe can thus be used for real-time monitoring of thiol (glutathione) level in specific tumor cells.

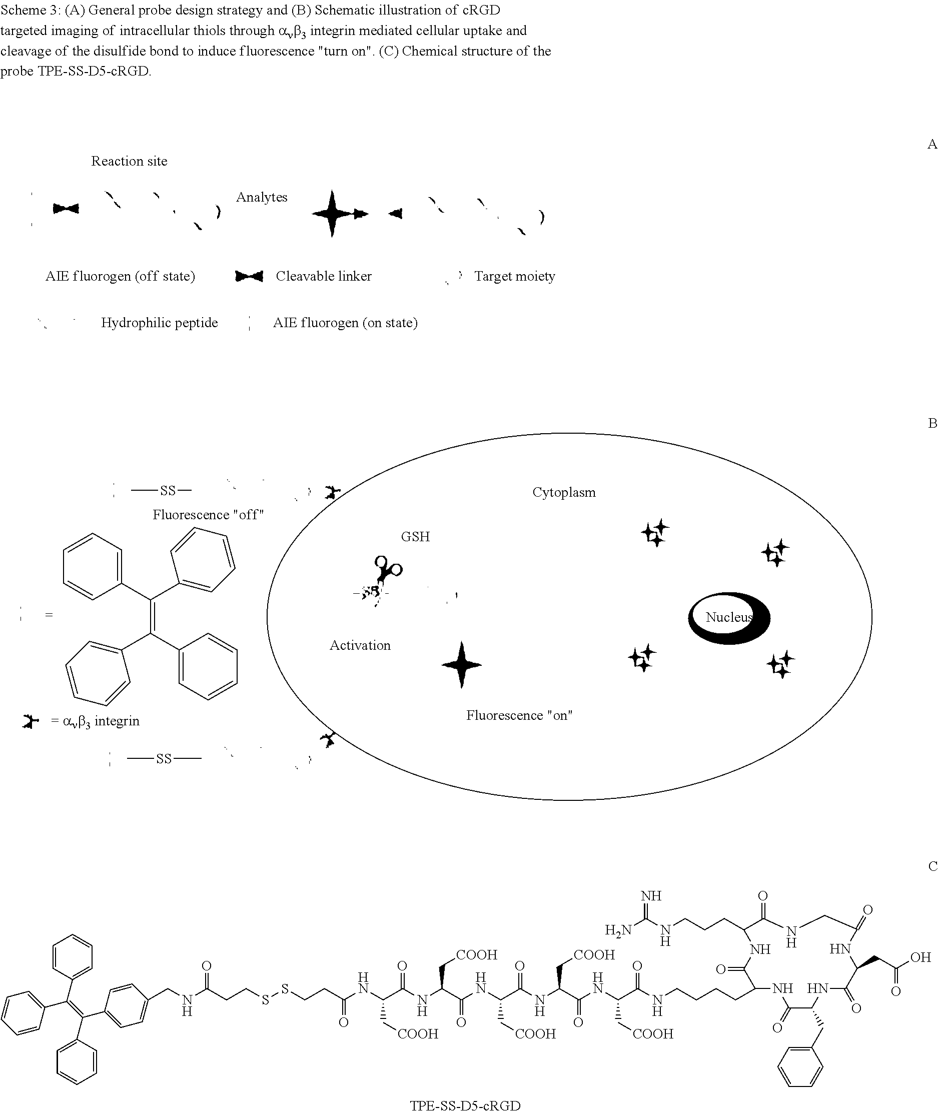

### Alkaline Phosphatase (ALP) Detection

The inventors of the present invention have developed a strategy for detecting alkaline phosphatase utilizing the light-up probe Phos-HC that has AIE-ESIPT characteristics.

An embodiment of this aspect of the present invention thus pertains to a method for the detection of alkaline phosphatase in a sample, comprising:

a) incubating a sample thought to comprise alkaline phosphatase with Phos-HC under conditions sufficient to form an incubated media; and

b) analyzing the incubated media of step a) by fluorescence spectroscopy,

wherein an increase in fluorescence intensity of a fluorescence signal at about 641 nm is indicative of the presence of alkaline phosphatase.

The sample of the method is preferably a live cell.

A more detailed description of this method of the present invention for detecting glutathione, including detection scheme and optical results, can be found in the Exemplification section of this application.

### EXEMPLIFICATION

### AIE Fluorogens

Detailed Synthetic Routes

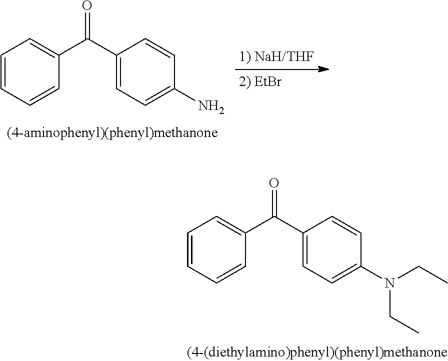 [SMILES: Nc1ccc(C(=O)c2ccccc2)cc1 ; CCN(CC)c1ccc(C(=O)c2ccccc2)cc1]

To a solution of (4-aminophenyl)(phenyl)methanone (1.970 g, 10 mmol) in THF (30 mL) was added sodium hydride (1.200 g, 30 mmol, 3 equiv, 60% suspension in oil) slowly at 0° C., the reaction was kept for 2 h, then bromoethane (2.24 mL, 30 mmol, 3 equiv) was injected. The reaction mixture was warmed slowly to room temperature and stirred overnight. The solution was diluted with dichloromethane and washed with aq. NaHCO₃ solution and brine. The organic layer was dried over sodium sulfate. The filtrate was concentrated and purified by silica gel column chromatography (ethyl acetate/hexane=1/10) to yield the yellow solid (2.302 g, 91%).

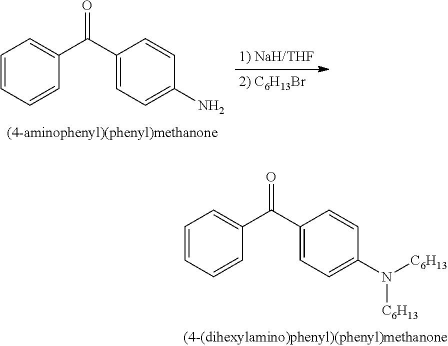

To a solution of (4-aminophenyl)(phenyl)methanone (0.986 g, 5 mmol) in THF (30 mL) was added sodium hydride (0.600 g, 15 mmol, 3 equiv, 60% suspension in oil) slowly at 0° C., the reaction was kept for 2 h, then n-bromohexane (2.476 g, 15 mmol, 3 equiv) was injected. The reaction mixture was warmed slowly to room temperature and stirred overnight. The solution was diluted with dichloromethane and washed with aq. NaHCO₃ solution and brine. The organic layer was dried over sodium sulfate. The filtrate was concentrated and purified by silica gel column chromatography (ethyl acetate/hexane=1/10) to yield the yellow solid (1.605 g, 88%).

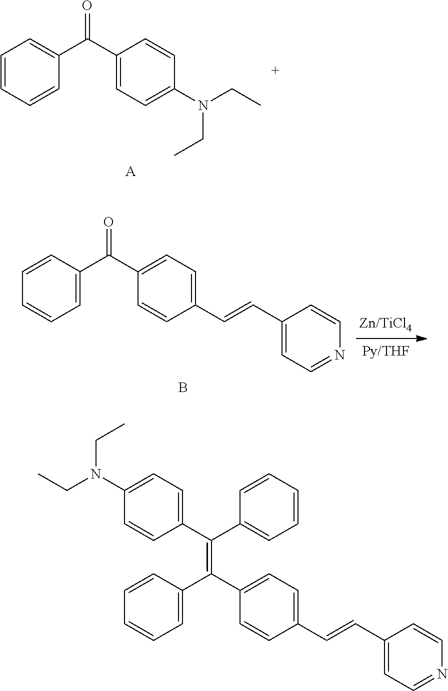

Under an Ar (g) atmosphere, a two-necked flask equipped with a magnetic stirrer was charged with zinc powder (1.308 g, 20 mmol) and 40 mL THF. The mixture was cooled to −5 to 0° C., and TiCl₄ (1.09 mL, 10 mmol) was slowly added by a syringe with the temperature kept under 10° C. The suspending mixture was warmed to room temperature and stirred for 0.5 h, then heated at reflux for 2.5 h. The mixture was again cooled to −5 to 0° C., charged with pyridine (0.5 mL, 6 mmol) and stirred for 10 min. The solution of A (506 mg, 2 mmol)+B (570 mg, 2 mmol) in 15 mL THF was added slowly. After addition, the reaction mixture was heated at reflux for overnight. The reaction was quenched with 10% K₂CO₃ aqueous solution and taken up with CH₂Cl₂. The organic layer was collected and concentrated. The crude material was purified by silica gel column chromatography (EA/DCM=2:5) to give the desire yellow products (0.360 g, 36%).

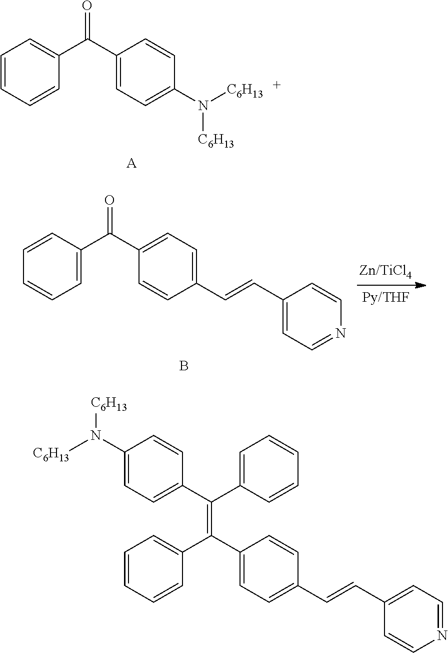

Under an Ar (g) atmosphere, a two-necked flask equipped with a magnetic stirrer was charged with zinc powder (1.308 g, 20 mmol) and 40 mL THF. The mixture was cooled to −5 to 0° C., and TiCl₄ (1.09 mL, 10 mmol) was slowly added by a syringe with the temperature kept under 10° C. The suspending mixture was warmed to room temperature and stirred for 0.5 h, then heated at reflux for 2.5 h. The mixture was again cooled to −5 to 0° C., charged with pyridine (0.5 mL, 6 mmol) and stirred for 10 min. The solution of A (731 mg, 2 mmol)+B (570 mg, 2 mmol) in 15 mL THF was added slowly. After addition, the reaction mixture was heated at reflux for overnight. The reaction was quenched with 10% K₂CO₃ aqueous solution and taken up with CH₂Cl₂. The organic layer was collected and concentrated. The crude material was purified by silica gel column chromatography (EA/DCM=2:5) to give the desire yellow products (0.360 g, 29%).

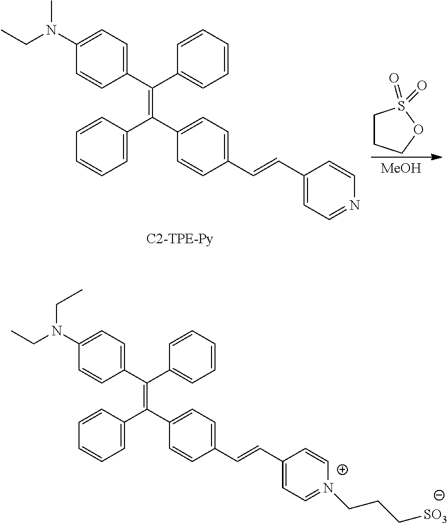

Under an Ar (g) atmosphere, a two-necked flask equipped with a magnetic stirrer was charged with C2-TPE-Py (100 mg, 0.197 mmol) and 1,3-Propanesultone (241 mg, 1.97 mmol) in methanol (10 mL). The reaction mixture was refluxed for 24 h, then solvent was removed under vacuum and the residue was subjected for silica gel column chromatography (methanol/DCM=1/3 to pure MeOH) to yield the yellow solid (91 mg, 73%).

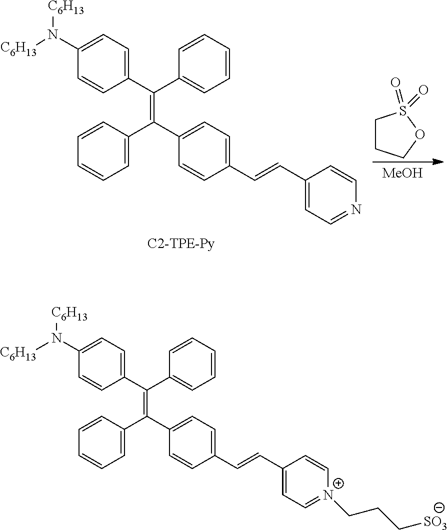

Under an Ar (g) atmosphere, a two-necked flask equipped with a magnetic stirrer was charged with C6-TPE-Py (62 mg, 0.1 mmol) and 1,3-Propanesultone (122 mg, 1.0 mmol) in methanol (10 mL). The reaction mixture was refluxed for 24 h, then solvent was removed under vacuum and the residue was subjected for silica gel column chromatography (methanol/DCM=1/3 to pure MeOH) to yield the yellow solid (61 mg, 82%).

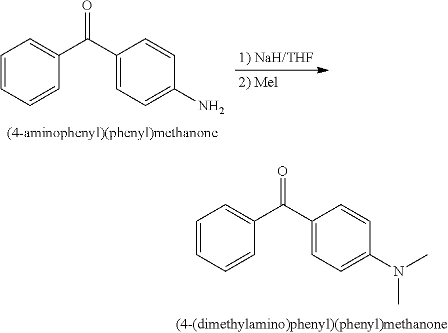 [SMILES: Nc1ccc(C(=O)c2ccccc2)cc1 ; CN(C)c1ccc(C(=O)c2ccccc2)cc1]

To a solution of (4-aminophenyl)(phenyl)methanone (1.970 g, 10 mmol) in THF (30 mL) was added sodium hydride (1.200 g, 30 mmol, 3 equiv, 60% suspension in oil) slowly at 0° C., the reaction was kept for 2 h, then iodomethane (1.87 mL, 30 mmol, 3 equiv) was injected. The reaction mixture was warmed slowly to room temperature and stirred overnight. The solution was diluted with dichloromethane and washed with aq. NaHCO₃ solution and brine. The organic layer was dried over sodium sulfate. The filtrate was concentrated and purified by silica gel column chromatography (ethyl acetate/hexane=1/5) to yield the yellow solid (2.010 g, 89%).

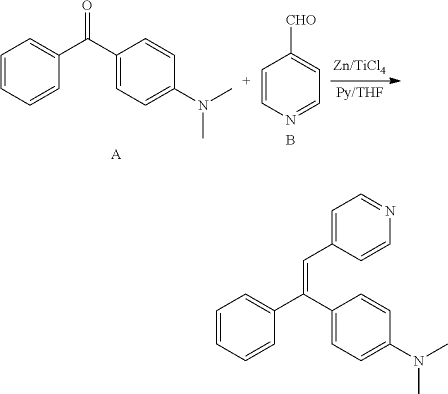

Under an Ar (g) atmosphere, a two-necked flask equipped with a magnetic stirrer was charged with zinc powder (2.616 mL, 20 mmol) and 40 mL THF. The mixture was cooled to −5 to 0° C., and TiCl₄ (2.16 mL, 20 mmol) was slowly added by a syringe with the temperature kept under 10° C. The suspending mixture was warmed to room temperature and stirred for 0.5 h, then heated at reflux for 2.5 h. The mixture was again cooled to −5 to 0° C., charged with pyridine (1.0 mL, 12 mmol) and stirred for 10 min. The solution of A (900 mg, 4 mmol)+B (805 mg, 4 mmol) in 30 mL THF was added slowly. After addition, the reaction mixture was heated at reflux for overnight. The reaction was quenched with 10% K₂CO₃ aqueous solution and taken up with CH₂Cl₂. The organic layer was collected and concentrated. The crude material was purified by silica gel column chromatography (EA/DCM=2:5) to give the desired yellow products (0.280 g, 23%).

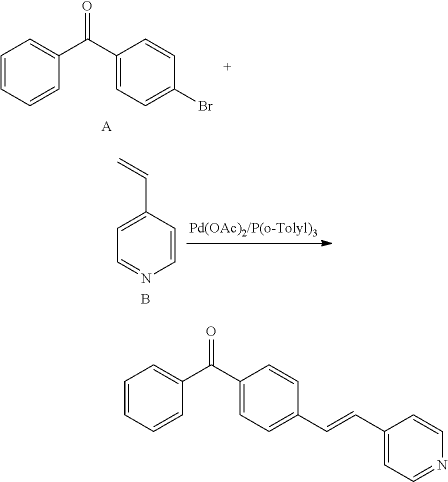

Under an Ar (g) atmosphere, a two-necked flask equipped with a magnetic stirrer was charged with A (2.616 g, 10 mmol), 4-vinylpyridine (1.25 mL, 11 mmol), Pd(OAc)₂ (90 mg, 4% mmol), P(o-Tolyl)₃ (426 mg, 14% mmol), Et₃N (36 mL), DMF (24 mL), were heated to 110° C. for 30 h. The reaction was then diluted with water and the aqueous phase was washed with CH₂Cl₂ and extracted with CHCl₃. Collecting all the organic layers, and evaporate the solvents, a yellow crude product was collected, then recrystallization (EA/CHCl₃) was performed, and the title product was achieved as yellow powder (2.600 g, 91%).

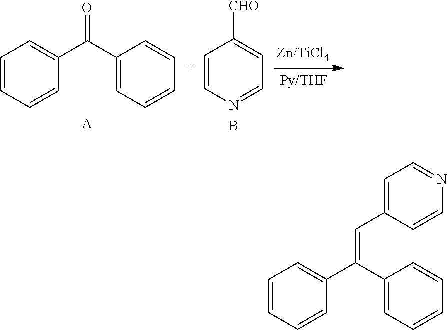

Under an Ar (g) atmosphere, a two-necked flask equipped with a magnetic stirrer was charged with zinc powder (2.616 mL, 20 mmol) and 40 mL THF. The mixture was cooled to −5 to 0° C., and TiCl₄ (2.16 mL, 20 mmol) was slowly added by a syringe with the temperature kept under 10° C. The suspending mixture was warmed to room temperature and stirred for 0.5 h, then heated at reflux for 2.5 h. The mixture was again cooled to −5 to 0° C., charged with pyridine (1.0 mL, 12 mmol) and stirred for 10 min. The solution of A (729 mg, 4 mmol)+B (805 mg, 4 mmol) in 30 mL THF was added slowly. After addition, the reaction mixture was heated at reflux for overnight. The reaction was quenched with 10% K₂CO₃ aqueous solution and taken up with CH₂Cl₂. The organic layer was collected and concentrated. The crude material was purified by silica gel column chromatography (EA/DCM=2:5) to give the desired yellow products (0.230 g, 22%).

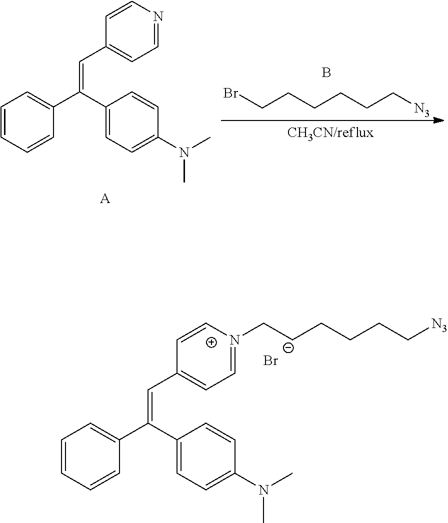

Under an Ar (g) atmosphere, a two-necked flask equipped with a magnetic stirrer was charged with A (100 mg, 0.33 mmol), B (1.00 g, 10 eq) in CH₃CN (10 mL), the reaction mixture was refluxed for at least 36 h. The reaction was quenched until the consumption of the starting material A. The solvent was evaporated and subjected for column chromatography (DCM, CH₃OH), the salt was achieved as a red solid (120 mg, 71%).

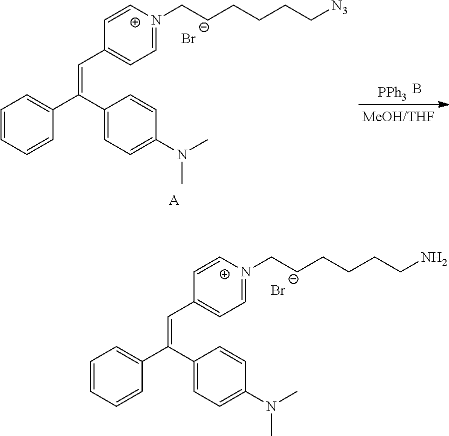

Under an Ar (g) atmosphere, a two-necked flask equipped with a magnetic stirrer was charged with A (50 mg, 0.10 mmol), B (52 mg, 2 eq) in a mixture of MeOH (10 mL) and THF (5 mL), the reaction mixture was refluxed for at least 48 h. The reaction was quenched until the consumption of the starting material A. The solvent was evaporated and subjected for column chromatography (DCM, CH₃OH), the salt was achieved as a red solid (the yield was not calculated).

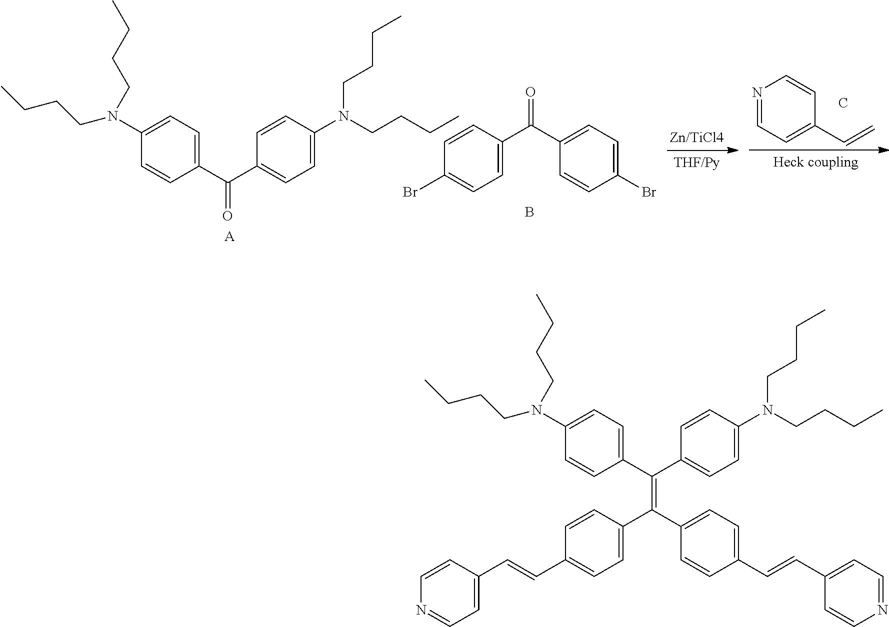

Under an Ar (g) atmosphere, a two-necked flask equipped with a magnetic stirrer was charged with zinc powder (1.308 g, 20 mmol) and 40 mL THF. The mixture was cooled to −5 to 0° C., and TiCl₄ (1.09 mL, 10 mmol) was slowly added by a syringe with the temperature kept under 10° C. The suspending mixture was warmed to room temperature and stirred for 0.5 h, then heated at reflux for 2.5 h. The mixture was again cooled to −5 to 0° C., charged with pyridine (0.5 mL, 6 mmol) and stirred for 10 min. The solution of A (870 mg, 2 mmol)+B (680 mg, 2 mmol) in 15 mL THF was added slowly. After addition, the reaction mixture was heated at reflux for overnight. The reaction was quenched with 10% K₂CO₃ aqueous solution and taken up with CH₂Cl₂. The organic layer was collected and concentrated. The mixed crude material was purified by silica gel column chromatography a yellow mixture. The mixture was placed in a sealed tube, and then charged with 4-vinylpyridine (0.62 mL, 5.5 mmol), Pd(OAc)₂ (45 mg, 4% mmol), P(o-Tolyl)₃ (213 mg, 14% mmol), Et₃N (12 mL), DMF (8 mL). The reaction was heated to 110° C. for 24 h. The reaction was then diluted with water and the aqueous phase was washed with CH₂Cl₂ and extracted with CHCl₃. The organic layer was collected and concentrated. The crude material was purified by silica gel column chromatography (EA/DCM=2:5) to give the desired yellow products (0.260 g, 16% in 2 steps).

### AIE-ESIPT Fluorogens

AIE Behavior of DMA-HC

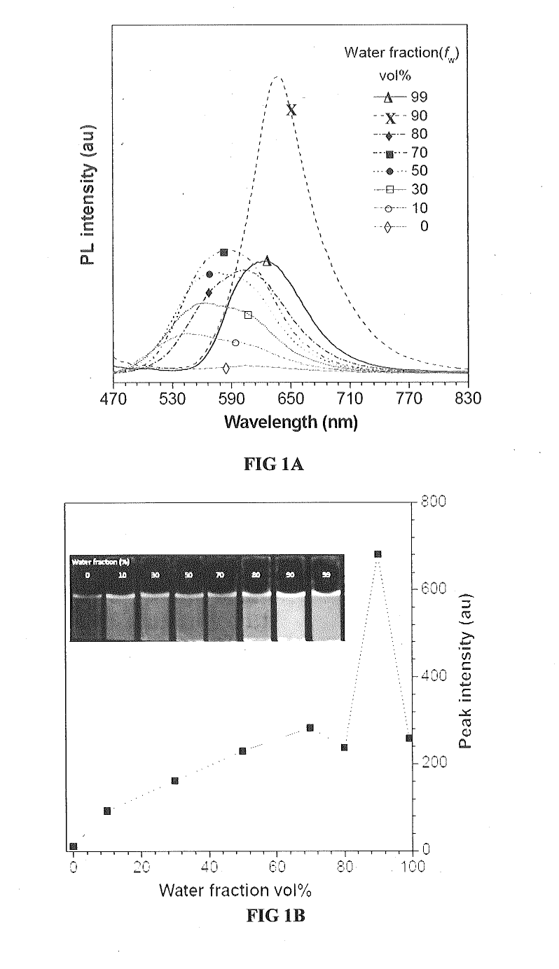

Upon addition of water to the THF solution of DMA-HC, the solution fluorescence red-shifts and intensifies upon aggregation formation. See FIGS. 1A-1B.

ALP Detection

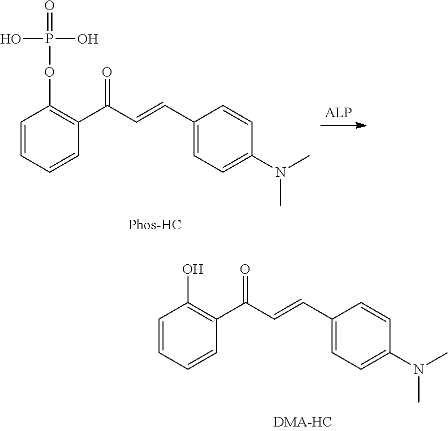 [SMILES: CN(C)c1ccc(/C=C/C(=O)c2ccccc2OP(=O)(O)O)cc1 ; CN(C)c1ccc(/C=C/C(=O)c2ccccc2O)cc1]

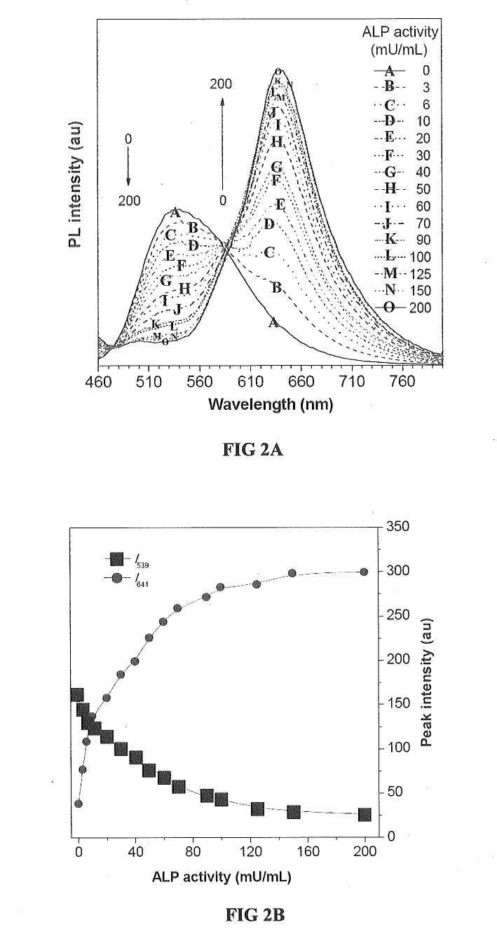

The probe-1 gives distinct optical response to ALP in solution. The emission maximum of the solution changes from 520 nm to 640 nm in the presence of ALP. See FIGS. 2A-2B.

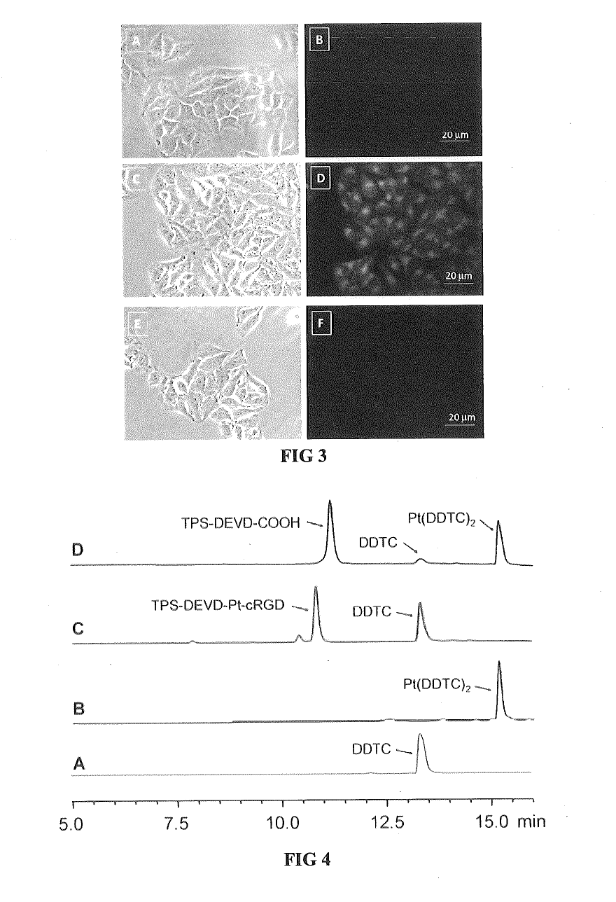

The same probe can also be used for cellular ALP detection. See FIG. 3.

The probe thus demonstrates a new strategy for AIE based light-up sensing and imaging.

### Light-Up Probe TPS-DEVD-Pt-cRGD

General Information

Cisplatin, N,N-diisopropylethylamine (DIEA), N-hydroxysuccinimide (NHS), 1-ethyl-3-[3-dimethylaminopropyl]carbodiimide hydrochloride (EDC), copper(II) sulfate (CuSO₄), sodium ascorbate, ascorbic acid, succinic anhydride, 3-(4,5-dimethylthiazol-2-yl)-2,5-diphenyltetrazolium bromide (MTT), anhydrous dimethyl sulfoxide (DMSO), anhydrous dimethylformamide (DMF), lithium wires, naphthalene, 4-bromobenzene, 4-bromobenzyl bromide, sodium azide, dichlorobis(triphenylphosphine)palladium(II), ZnCh.TMEDA, piperazine-N,N′-bis(2-ethanesulfonic acid) (PIPES), diethyldithiocarbamate (DDTC), bovine serum albumin (BSA), lysozyme, pepsin, trypsin and other chemicals were all purchased from Sigma-Aldrich and used as received without further purification. Hexane and tetrahydrofuran (THF) purchased from Fisher Scientific were distilled from sodium benzophenoneketyl immediately prior to use. Dichloromethane (DCM) was distilled over calcium hydride. Deuterated solvents with tetramethylsilane (TMS) as internal reference were purchased from Cambridge Isotope Laboratories Inc. Alkyne-functionalized DEVD (Asp-GluVal-Asp-Pra) and amine-functionalized cRGD (cyclic(Arg-Gly-Asp-D-Phe-Lys)) were customized from GL Biochem Ltd. cis,cis,trans-Diamminedichlorodisuccinatoplatinum(IV) was synthesized following a literature method. [1]

Dulbecco's modified essential medium (DMEM) was a commercial product of National University Medical Institutes (Singapore). Milli-Q water was supplied by Milli-Q Plus System (Millipore Corporation, Breford, USA). Piperazine-N,N′-bis(2-ethanesulfonic acid) (PIPES) buffer containing 50 mM PIPES, 100 mM NaCl, 1 mM ethylenediaminetetraacetic acid (EDTA), 0.1% w/v 3-[(3-cholamidopropyl)dimethylammonio]propanesulfonic and 25% w/v sucrose (pH=7.2). Recombinant human caspase-3 was purchased from R&D Systems. Caspase-3 inhibitor 5-[(S)-(+)-2-(methoxymethyl)pyrrolidino]sulfonylisatin was purchased from Calbiochem. Fetal bovine serum (FBS) and trypsin-EDTA solution were purchased from Gibco (Lige Technologies, AG, Switzerland). Staurosporine (STS) was purchased from Biovision. DRAQ5 was purchased from Biostatus. Cleaved caspase-3 (Asp 175) (5A1E) rabbit mAb (#9664) was purchased from Cell Signaling. Mouse anti-rabbit IgG-TR (sc-3917) was purchased from Santa Cruz.

Characterization

NMR spectra were measured on a Bruker ARX 400 NMR spectrometer. Chemical shifts were reported in parts per million (ppm) referenced with respect to residual solvent (CDCl₃=7.26 ppm, (CD₃)₂SO=2.50 ppm or tetramethylsilane Si(CH₃)₄=0 ppm). Particle size and size distribution were determined by laser light scattering (LLS) with a particle size analyzer (90 Plus, Brookhaven Instruments Co., USA) at a fixed angle of 90° at room temperature. HPLC profiles and mass spectra were acquired using a Shimadzu IT-TOF. A 0.1% TFA/H₂0 and 0.1% TFA/acetonitrile were used as eluents for the HPLC experiments. High resolution mass spectra (HRMS) were recorded on a Finnigan MAT TSQ 7000 Mass Spectrometer. UV-vis absorption spectra were taken on a Milton Ray Spectronic 3000 array spectrophotometer. Photoluminescence (PL) spectra were measured on a Perkin-Elmer LS 55 spectrofluorometer. The cells were imaged by confocal laser scanning microscope (CLSM, Zeiss LSM 410, Jena, Germany) with imaging software (Fluoview FV500). The images were analyzed by Image J 1.43 x program (developed by NIH, http://rsbweb.nih.gov/ij/).

### Synthesis of 4-Bromobenzyl Azide

Into a flask equipped with a magnetic stirrer were added 4-bromobenzyl bromide (7.5 g, 30 mmol), sodium azide (7.8 g, 120 mmol) and 40 mL of DMSO. After stirring at 70° C. for 12 h, the solution was poured into 150 mL of water and extracted with DCM. The crude product was purified by silica-gel chromatography using hexane as eluent to give a colorless viscous liquid in 96% yield (6.12 g). ¹H NMR (CDCl₃, 400 MHz), δ (TMS, ppm): 7.47 (d, 2H), 7.15 (d, 2H), 4.26 (s, 2H). ¹³C NMR (CDCl₃, 100 MHz), δ (TMS, ppm): 134.3, 131.8, 129.6, 122.1, 53.9. HRMS (MALDI-TOF): m/z 210.9640 (M⁺, calcd 210.9745).

### Synthesis of 1,1-dimethyl-2-[4-(azidomethyl)phenyl]-3,4,5-triphenylsilole (TPS-CH₂N₃)

Dimethylbis(phenylethynyl)silane was prepared according to our published procedures. [2] A mixture of lithium (0.056 g, 8 mmol) and naphthalene (1.04 g, 8 mmol) in 8 mL of THF was stirred at room temperature under nitrogen for 3 h to form a deep dark green solution of LiNaph. A solution of dimethylbis(phenylethynyl)silane (0.52 g, 2 mmol) in 5 mL of THF was then added dropwise to LiNaph solution at room temperature. After stirring for 1 h, the mixture was cooled to 0° C. and then diluted with 25 mL of THF. A black suspension was formed upon addition of ZnCl₂.TMEDA (2 g, 8 mmol). After stirring for an additional hour at room temperature, a solution containing 4-bromobenzene (0.34 g, 2.2 mmol), 4-bromobenzyl azide (0.47 g, 2.2 mmol) and PdCl₂(PPh₃)₂ (0.08 g, 0.1 mmol) in 25 mL of THF was added. The mixture was refluxed overnight. After cooling down to room temperature, 100 mL of 1M HCl solution was added and the mixture was extracted with DCM several times. The organic layer was combined and washed with brine and water and then dried over magnesium sulfate. After solvent evaporation under reduced pressure, the residue was purified by a silica-gel column using hexane as eluent. The product was obtained as a yellow solid in 36% yield (0.34 g). ¹H NMR (400 MHz, CDCl₃), δ (TMS, ppm): 7.15-7.06 (m, 6H), 7.02-6.99 (m, 5H), 6.95-6.93 (m, 4H), 6.82-6.79 (m, 4H), 4.23 (s, 2H), 0.48 (s, 6H). ¹³C NMR (CDCl₃, 100 MHz), δ (TMS, ppm): 154.5, 153.9, 142.1, 141.2, 140.1, 139.8, 138.7, 132.5, 130.0, 129.2, 128.9, 128.0, 127.5, 126.4, 126.3, 125.7, 54.7, −3.80. HRMS (MALDI-TOF): m/z 469.1959 (M⁺, calcd 469.1974).

### Synthesis of N-Hydroxysuccinimide-Activated Platinum (IV) Complexes

A mixture of platinum(IV) complex cis,cis,trans-diamminedichlorodisuccinatoplatinum(IV) (32.1 mg, 0.06 mmol), EDC (23.0 mg, 0.12 mmol) and NHS (13.8 mg, 0.12 mmol) in anhydrous DMF (1 mL) was stirred at room temperature overnight. After that, the mixture was purified by HPLC (solvent A: water with 0.1% TFA, solvent B: CH₃CN with 0.1% TFA) and quickly lyophilized to yield the desired product as a white powder in 78% yield (34.1 mg). ¹H NMR (400 MHz, DMF-d₇), δ (TMS, ppm): 6.92-6.68 (m, 6H), 2.94-2.91 (m, 8H), 2.89-2.84 (m, 4H), 2.72-2.68 (m, 4H). ¹³C NMR (DMF-d₇, 100 MHz), δ (TMS, ppm): 178.5, 170.6, 168.8, 30.0, 27.1, 25.9. IT-TOF-MS: m/z [M+H]⁺ calc. 728.026. found 728.021.

### “Click” Synthesis of the Apoptosis Sensor TPS-DEVD-NH₂

Alkyne-functionalized DEVD (10.2 mg, 20 μmol) and TPS-CH₂N₃ (9.4 mg, 20 μmol) were dissolved in a mixture of DMSO/H₂O solution (v/v=1/1; 1.0 mL). The “click” reaction was initiated by sequential addition of catalytic amounts of CuSO₄ (9.6 mg, 6 mop and sodium ascorbate (2.4 mg, 12 μmol). The reaction was continued with shaking at room temperature for another 24 h. The final product was purified by HPLC and lyophilized under vacuum to yield the probe as white powders in 45% yield (9.4 mg). ¹H NMR (DMSO-d₆, 400 MHz): 12.24 (s, 3H), 8.49 (d, 1H), 8.32 (d, 1H), 8.05 (d, 1H), 7.92 (d, 1H), 7.86 (s, 1H), 7.22-7.06 (m, 6H), 7.02-6.99 (m, 5H), 6.95-6.88 (m, 4H), 6.82-6.79 (m, 4H), 5.45 (s, 2H), 4.54-4.49 (m, 1H), 4.38 (m, 2H), 4.17-4.13 (m, 2H), 3.10-3.05 (m, 1H), 2.92-2.88 (m, 1H), 2.71-2.65 (m, 2H), 2.26-2.21 (m, 2H), 2.01-1.85 (m, 3H), 0.84-0.74 (m, 6H), 0.43 (s, 6H); IT-TOF-MS: m/z [M+H]⁺ calc. 1040.426. found 1040.866.

### Synthesis of Theranostic Prodrug TPS-DEVD-Pt-cRGD

TPS-DEVD-NH₂ (9.0 mg, 8.7 mmol) and amine-functionalized cRGD (5.2 mg, 8.7 mmol) were dissolved in anhydrous DMSO (1.0 mL) with a catalytic amount of DIEA (1.0 μL). The mixture was stirred at room temperature for 10 min. Then N-hydroxysuccinimide-activated platinum(IV) complex (6.3 mg, 8.7 mmol) in DMSO (0.5 mL) was added quickly to the above mixture. The reaction was continued with stirring at room temperature for another 24 h. The final product was purified by HPLC and lyophilized under vacuum to yield the prodrug as white powders in 40% yield (7.4 mg). ¹H NMR (DMSO-d₆, 400 MHz): 12.24 (s, 3H), 8.55 (d, 1H), 8.51 (d, 1H), 8.31 (d, 1H), 8.15-8.05 (m, 6H), 7.91 (d, 1H), 7.86 (s, 1H), 7.62 (d, 2H), 7.56 (d, 1H), 7.45 (m, 1H), 7.22-7.06 (m, 6H), 7.02-6.99 (m, 5H), 6.95-6.88 (m, 4H), 6.82-6.79 (m, 4H), 6.60-6.35 (m, 6H), 5.45 (s, 2H), 4.65-4.60 (m, 1H), 4.54-4.48 (m, 1H), 4.40-4.32 (m, 3H), 4.17-4.10 (m, 3H), 4.05-4.02 (m, 1H), 3.95-3.91 (m, 1H), 3.10-3.06 (m, 4H), 2.95-2.87 (m, 2H), 2.85-2.78 (m, 2H), 2.75-2.62 (m, 6H), 2.50-2.45 (m, 8H), 2.27-2.23 (m, 4H), 1.93-1.89 (m, 4H), 1.72 (m, 3H), 1.41-1.38 (m, 2H), 1.37-1.32 (m, 2H), 0.84-0.74 (m, 6H), 0.43 (s, 6H); ESI-MS: m/z [M+H]⁺ calc. 2141.719. found 2141.689.

General Procedure for Enzymatic Assay

DMSO stock solutions of TPS-DEVD-Pt-cRGD were diluted with a mixture of DMSO and PIPES (v/v=1/199) to 10 μM. Next, each probe was incubated with ascorbic acid or caspase-3 at room temperature and the change of fluorescence intensity was measured. The PL spectra were collected from 420 to 650 nm under an excitation wavelength at 365 nm.

Cell Culture

U87-MG human glioblastoma cancer cells, MCF-7 breast cancer cells and 293T normal cells were provided by American Type Culture Collection (ATCC). The cells were cultured in DMEM (Invitrogen, Carlsbad, Calif.) containing 10% heat-inactivated FBS (Invitrogen), 100 U/mL penicillin, and 100 μg/mL streptomycin (Thermo Scientific) and maintained in a humidified incubator at 37° C. with 5% C0₂. Before experiment, the cells were pre-cultured until confluence was reached.

Confocal Imaging

U87-MG, MCF-7 and 293T cells were cultured in the chambers (LAB-TEK, Chambered Coverglass System) at 37° C. After 80% confluence, the culture medium was removed and washed twice with PBS buffer. The probe in DMSO stock solution was then added to the chamber to reach a final concentration of 5 μM. In some experiments, the cells were pre-incubated with media containing cRGD (50 μM) or inhibitor (5 μM) prior to prodrug incubation. After incubation the prodrug at 37° C. for 2 h, the medium was replaced with fresh medium, after that the cells were washed twice with ice-cold PBS and the cell nucleus was living stained with DRAQ5 (Biostatus) following the standard protocol of the manufacturer. For co-localization with active caspase-3 antibody, the cells were first fixed for 15 min with 3.7% formaldehyde in 1×PBS at room temperature, washed twice with cold PBS again, and permeabilized with 0.1% Triton X-100 in PBS for 10 min. The cells were then blocked with 2% BSA in 1×PBS for 30 min and washed twice with PBS. The cells were subsequently incubated with a mixture of anti-caspase-3 antibody/PBS (v/v=1/99) for 1 h at, room temperature, washed once with PBS buffer, and then incubated with mouse anti-rabbit IgG-TR (0.8 μg mL⁻¹) in PBS for I h, following by washing with PBS again. The cells were then imaged immediately by confocal laser scanning microscope (CLSM, Zeiss LSM 410, Jena, Germany). The images were analyzed by Image J 1.43 x program (developed by NIH, http://rsbweb.nih.gov/ij/).

Quantification of Cell Apoptosis by Fluorescence Microplate Reader

U87-MG and MCF-7 cells were seeded in 96-well plates (Costar, USA) at an intensity of 4×104 cells mL⁻¹. After confluence, the medium was replaced by different concentrations of TPS-DEVD-Pt-cRGD in fresh PBS-free DMEM medium. After the determined incubation time at 37° C., the adherent cells were washed twice with 1×PBS buffer followed by fluorescence measurement using a T-CAN microplate reader. The excitation and emission wavelengths are 365 and 480 nm, respectively.

Cytotoxicity of the Prodrug

3-(4,5-Dimethythiazol-2-yl)-2,5-diphenyltetrazolium bromide (MTT) assays were used to assess the metabolic activity of U87-MG and MCF-7 cancer cells. The cells were seeded in 96-well plates (Costar, Ill., USA) at an intensity of 4×10⁴ cells mL⁻¹. After 24 h incubation, the medium was replaced by the probe suspension at different concentration of the prodrug and incubated at 37° C. After the designated time intervals, the wells were washed twice with 1×PBS buffer, and 100 μL of freshly prepared MTT (0.5 mg mL⁻¹) solution in culture medium was added into each well. The MTT medium solution was carefully removed after 3 h incubation in the incubator at 37° C. DMSO (100 μL) was then added into each well and the plate was gently shaken to dissolve all the precipitates formed. The absorbance of MTT at 570 nm was monitored by the microplate reader (Genios Tecan). Cell viability was expressed by the ratio of absorbance of the cells incubated with probe suspension to that of the cells incubated with culture medium only.

### Results Discussion

### Synthesis and Characterization of the Theranostic Platinum(IV) Prodrug TPS-DEVD-Pt-cRGD

Azide-functionalized tetraphenylsilole (TPS-CH₂N₃) was synthesized by the heterobifunctional modification of dimethylbis(phenylethynyl)silane with 4-bromobenzene and 4-bromobenzyl azide. Detailed synthesis and characterization of TPS-CH₂N₃ and the intermediates are shown in the Experimental Section and Supporting Information. The coupling between TPS-CH₂N₃ and alkyne-functionalized DEVD via “click” reaction using CuS0₄/sodium ascorbate as the catalyst in DMSO/water (v/v=1/1) afforded the apoptosis sensor TPS-DEVD-NH₂ in 45% yield after HPLC purification. The purity and identity of the probe was well characterized by analytical HPLC, NMR, and HRMS. Commercially available anticancer drug cisplatin was modified to be used as the linker between TPS-DEVD-NH₂ and the amine functionalized cRGD. In the first step, cisplatin was oxidized by hydrogen peroxide to produce cis, cis, trans-diaminedichlorodihydroxyplatinum(IV) complex. Next, the Pt(IV) complex was reacted with succinic anhydride in DMSO at 70° C. for 12 h to yield cis, cis, trans-diamminedichlorodisuccinatoplatinum(IV) complex. The activated Pt(IV) complex was subsequently obtained by reacting the carboxylic acid groups with NHS in anhydrous DMF using EDC as the coupling reagent. The activated Pt(IV) linker was purified by HPLC and lyophilized as white powder with a yield of 78%. Asymmetric functionalization of activated Pt(IV) linker with TPS-DEVD-NH₂ and aminefunctionalized cRGD in the presence of N,N-diisopropylethylamine (DIEA) in anhydrous DMSO afforded the desired product, TPS-DEVD-Pt-cRGD in 40% yield after HPLC purification (Scheme 4). The purity and identity of the probe was well characterized by HPLC, NMR, and HRMS.

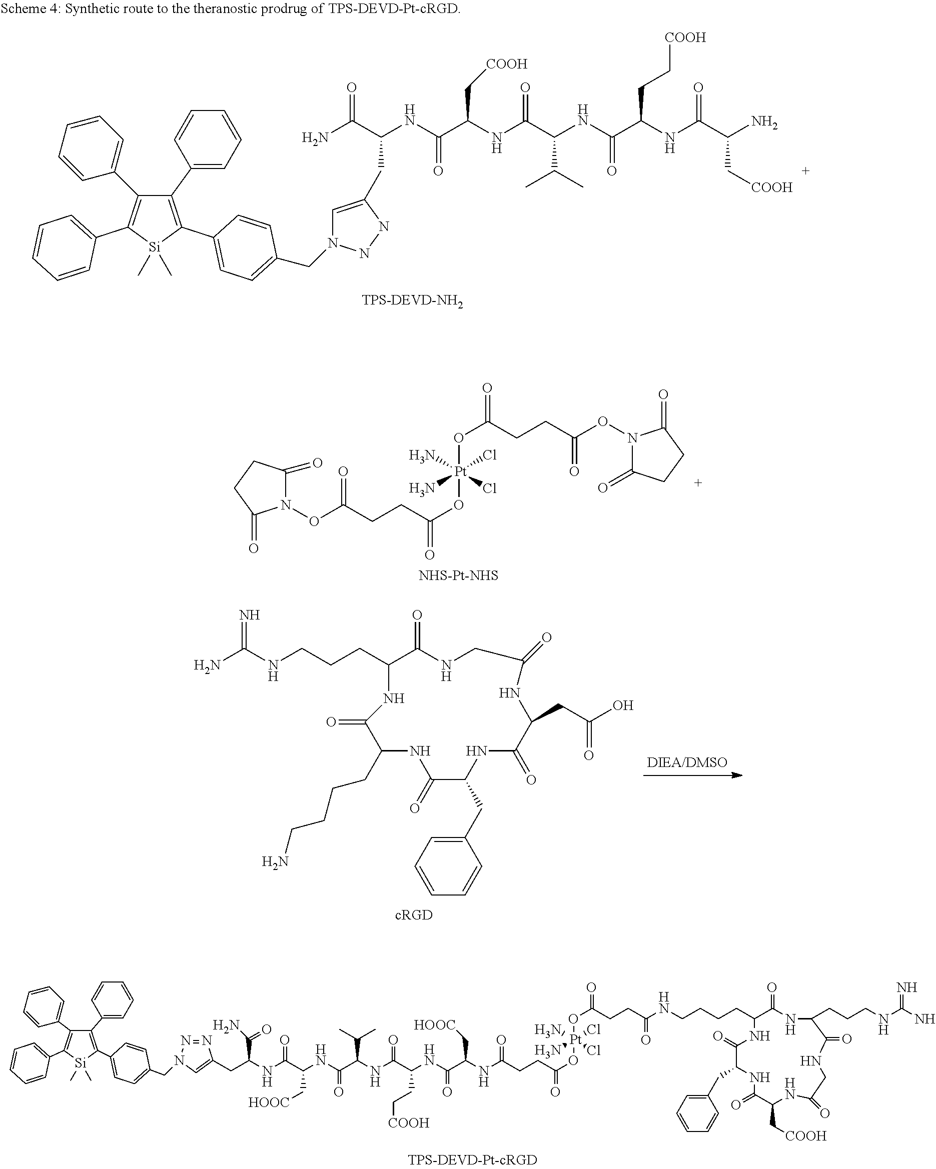

For any prodrug, it is essential that it can be easily transformed into its original form to restore its therapeutic ability after the modification. To evaluate our prodrug as a potential drug delivery system, we studied the nature of the formed Pt(II) species upon reduction of the synthesized prodrug. It is reported that diethyldithiocarbamate (DDTC) can react with Pt(II) complexes to yield the adducts Pt(DDTC)₂ but does not react with stable Pt(IV) complexes. [3][4] In this work, we use HPLC-MS system to monitor the adducts formation of Pt(IV) complexes before and after the reduction with ascorbic acid in the presence of DDTC. We choose ascorbic acid as a reduction agent because it is highly abundant in the cells (1 mM), which has been demonstrated to be a major substance for the reduction of Pt(IV). [5] As shown in FIG. 4, cisplatin can efficiently bind to DDTC to form the adduct of Pt(DDTC)₂. The complex was further confirmed by IT-TOF with a mass-to-charge ratio (m/z) of 492.104. On the other hand, Pt(DDTC)₂ can only be formed when the prodrug is treated with ascorbic acid and in the presence of the DDTC, confirming that the released Pt entities were indeed Pt(II) species. In addition, an apoptosis sensor TPS-DEVD-COOH with an m/z of 1140.344 is formed after the reduction. Based on these results, we confirm that the Pt(IV) prodrug can be reduced in the presence of ascorbic acid to generate the reactive Pt(II) drug and apoptosis sensor simultaneously.

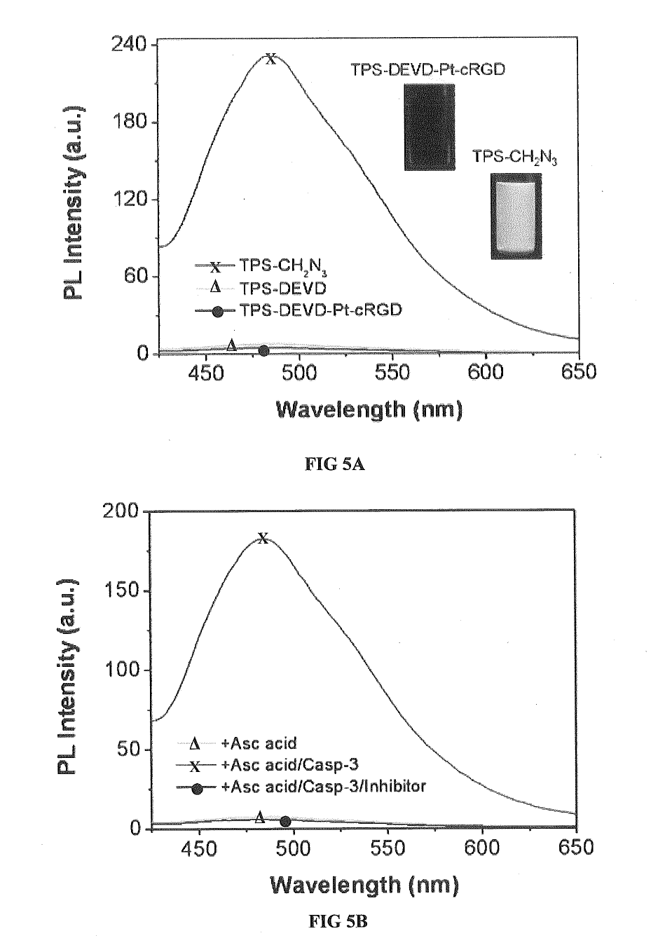

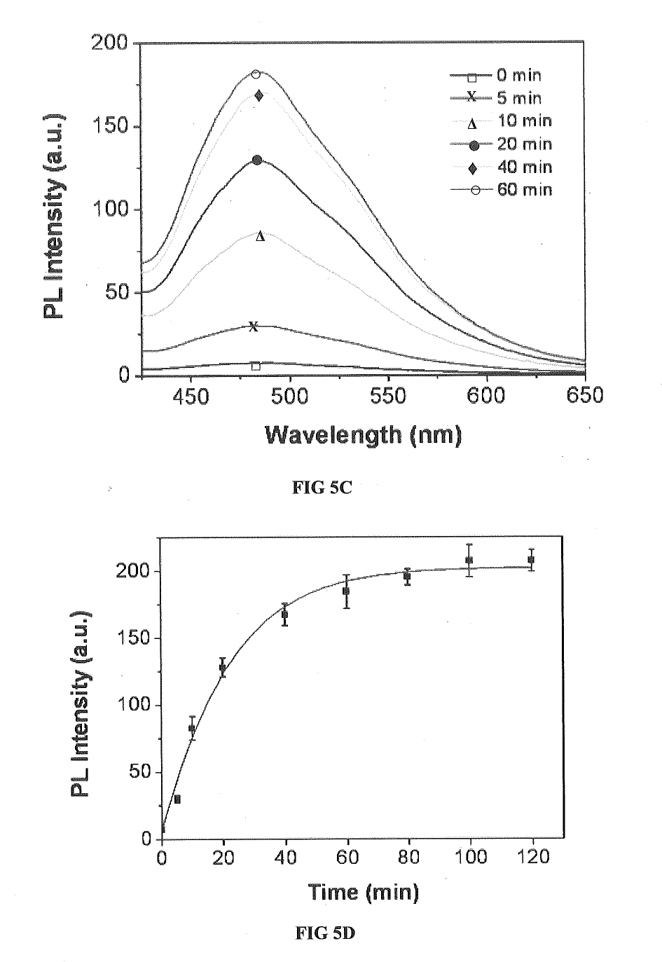

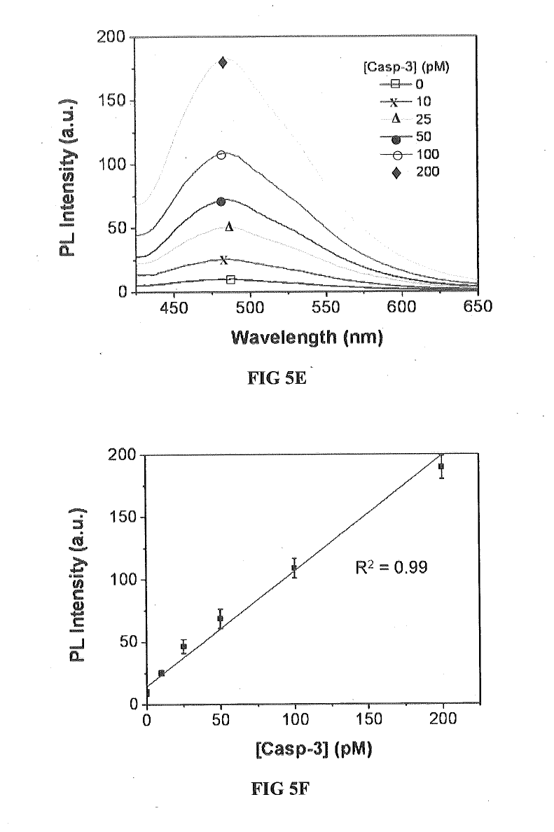

We next studied the optical properties of our prodrug. The UV-vis absorption spectra of TPS-CH₂N₃ in THF and TPS-DEVD-Pt-cRGD in DMSO/PIPES (v/v=1/199) buffer were obtained. Both have similar absorption profiles with an obvious absorbance in the 320-440 nm range with a little blue shift after the modification of the AIE fluorogen. It is known that AIE fluorogen is non-fluorescent in good solvents but emits intensely in solid or as aggregates in poor solvents. [6][7] As can be seen from the photoluminescence (PL) spectra shown in FIG. 5A, TPS-CH₂N₃ shows intense fluorescence in a mixture of DMSO/PIPEs (v/v=1/199), while the TPS-DEVD-NH₂ and TPS-DEVD-Pt-cRGD are almost non-fluorescent in the same medium, due to their good solubility in water. The aggregate formation for hydrophobic TPS-CH₂N₃ in a mixture of DMSO/PIPEs (v/v=1/199) buffer was confirmed by laser light scattering (LLS) measurement, which shows an average diameter of 118 nm. As biosensing is often conducted in buffers, it is important to study the effect of ionic strength on the emission behavior of the prodrug. The experiments were performed with addition of sodium chloride into an aqueous solution of TPS-DEVD-Pt-cRGD (10 μM). Almost no change in fluorescent intensity of the probe is observed when the concentration of NaCl is increased from 0 to 960 mM. Clearly, ionic strength does not affect the fluorescent property of TPS-DEVD-Pt-cRGD and its reduction product. Its PL profile also does not change in the commonly used medium Dulbecco's Modified Eagle Medium (DMEM). TPS-DEVD-Pt-cRGD and its reduction product maintain an “off” state in the complex environment and thus have great potential to serve as a specific light-up apoptosis sensor for drug effect study with minimum background interference.

First, we demonstrated that the fluorescence of TPS-DEVD-NH₂ in DMSO/PIPEs (v/v=1/199) increased upon the addition of caspase-3 for 60 min. FIG. 5B shows optical properties of the prodrug after reduction with ascorbic acid. The fluorescence does not change after the reduction of TPS-DEVD-Pt-cRGD to TPS-DEVD-COOH. To study the enzymatic response of TPS-DEVD-COOH, we performed in vitro enzymatic assays with recombinant human caspase-3. Mixtures of the ascorbic acid pretreated prodrug (10 μM) and caspases-3 (200 pM) were prepared and incubated in PIPES buffer. After 1 h incubation, the PL spectra were measured in the range from 425 to 650 nm. As shown in FIG. 5B, strong fluorescence signals are recorded for ascorbic acid pretreated TPS-DEVD-Pt-cRGD upon treatment with caspase-3. However, most of the fluorescence is readily competed away by pretreatment of the probe with 5-[(S)-(+)-2-(methoxymethyl)pyrrolidino]sulfonylisatin, a highly specific inhibitor of caspase-3, [8] indicating that specific cleavage of DEVD from TPS-DEVD-COOH is inhibited. This caspase-3 catalyzed hydrolysis was further confirmed by LC-MS with the formation of TPS residue with an m/z of 582.659. After treatment with caspase-3, particles with an average diameter of 109 nm are formed for the TPS residue, which explains the solution fluorescence “turn on”.

The fluorescence change of the TPS-DEVD-Pt-cRGD solution (10 μM) upon addition of ascorbic acid and caspase-3 enzyme was also monitored over time in a mixture of DMSO/PIPEs (v/v=1/199) buffer. As shown in FIG. 5C, a quick fluorescence increase in solution is observed after incubation with caspase-3. The fluorescence reaches a plateau in 60 min which is 28-fold higher than its intrinsic emission (FIG. 5D). We further studied the effect of caspase-3 concentration on the solution emission. Different concentrations of caspase-3 ranging from 0 to 200 pM were incubated in a mixture of DMSO/PIPEs (v/v=1/199) buffer with TPS-DEVD-Pt-cRGD (10 μM) for 1 h, and the corresponding spectra are shown in FIG. 5E. With increasing concentrations of caspase-3, the PL intensities gradually enhanced due to the increased amount of TPS aggregates formed in aqueous media. As shown in FIG. 5F, the plot of PL intensities at 485 nm against the caspase-3 concentration gives a perfect linear line (R²=0.99), suggesting the possibility of quantification of caspase-3 on the basis of the PL intensity changes. The detection limit for caspase-3 is estimated to be 1 pM based on three times sigma method.

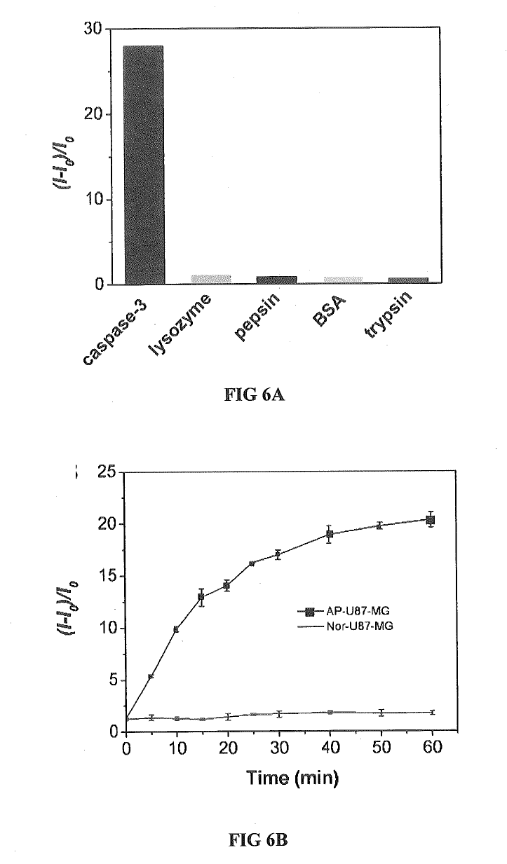

To study the selectivity of the prodrug, we incubated ascorbic acid pretreated TPS-DEVD-Pt-cRGD (10 μM) with several proteins, including lysozyme, pepsin, bovine serum albumin (BSA) and trypsin under identical conditions. As shown in FIG. 6A, only caspase-3 displays a 28-fold fluorescence increase while the intensities from other proteins remain low, confirming that DEVD is specifically recognized and cleaved by caspase-3. This result demonstrates that our probe can be used as a specific indicator of the caspase-3 in the cells. As there are many different kinds of proteins or enzymes inside the cells, we further obtained the cellular lysate of normal and apoptotic U87-MG cells, which were pretreated with commonly used cell apoptosis inducer staurosporine (STS, 2 μM) to activate the caspase-3 enzyme in the cells. [9] The cell lysates were directly incubated with TPS-DEVD-Pt-cRGD (5 μM) and the fluorescence intensity at 485 nm was monitored over time. As shown in FIG. 6B, the fluorescence intensity increases quickly in a similar way to that of the solution study with caspase-3 in FIG. 5C. Meanwhile, the fluorescence intensity at 485 nm shows a minimum change after incubation with the lysate of normal cells indicating that the prodrug is highly stable upon treatment with cellular proteins except caspase-3.

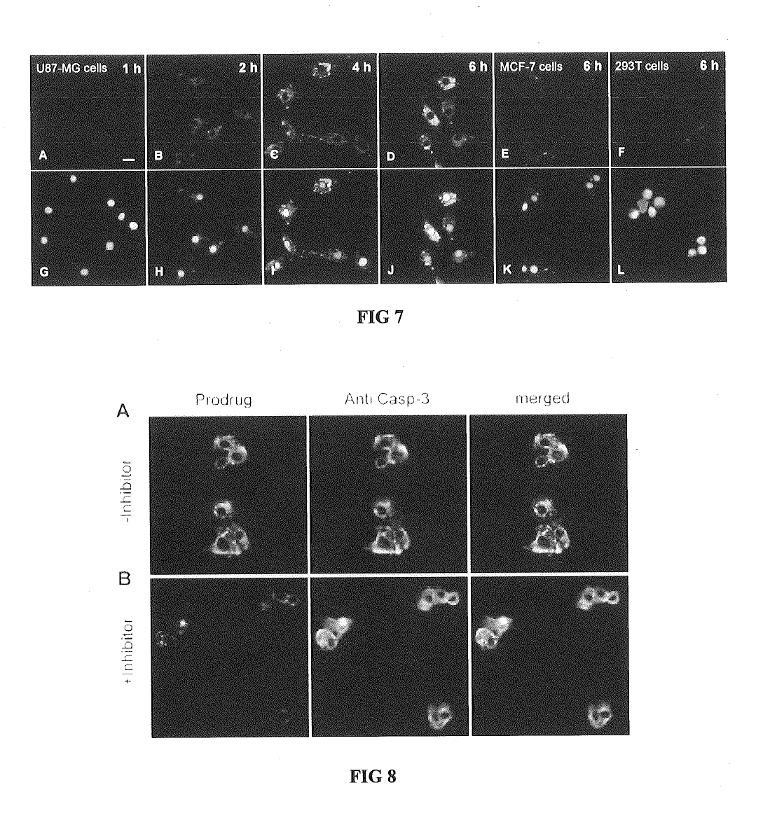

To explore the capability of using TPS-DEVD-Pt-cRGD as a targeted drug delivery system and a drug induced apoptosis imaging probe in cancer cells. We first incubated TPS-DEVD-NH₂ with U87-MG cells at 37° C. After 2 h of incubation, the cells were treated with cisplatin or staurosporine which both can activate the cell apoptosis and monitored with confocal microscopy. It was seen that the normal, un-induced cells show very low fluorescence intensity, indicating little or no caspase-3 activity. In sharp contrast, strong fluorescence signals are collected from the cells treated with cisplatin or staurosporine. These results demonstrate that TPS-DEVD-NH₂ can be used as an indicator of cell apoptosis. We next incubated the prodrug TPS-DEVD-Pt-cRGD with U87-MG human glioblastoma, MCF-7 breast cancer cell lines and normal cell line 293T cells, the confocal imaging results are shown in FIG. 7. U87-MG cells with overexpressed integrin α₁β₃ on cellular membrane was chosen as integrin-positive cancer cell, while MCF-7 and 293T cells with low integrin expression were used as the negative control. After incubation with TPS-DEVD-Pt-cRGD, realtime imaging experiments of all cells were performed. As the incubation time elapses, the fluorescence of U87-MG increases gradually with the cellular apoptotic progress, which reaches a maximum at 6 h. On the contrary, only a weak fluorescence signal could be found for MCF-7 and 293T cells even after 6 h incubation. When U87-MG cells were pre-treated with free cRGD or/and caspase-3 inhibitor prior to TPS-DEVD-Pt-cRGD incubation, the image showed weak fluorescence. These results clearly demonstrate that TPS-DEVD-Pt-cRGD not only can be used for targeted drug delivery but also has the potential for real-time monitoring of caspase-3 activity in situ. Furthermore, excellent overlap is observed between the confocal images of the probe and immunofluorescence signals generated from anti-caspase-3 primary antibody and a Texas Red-labeled secondary antibody (FIG. 8).

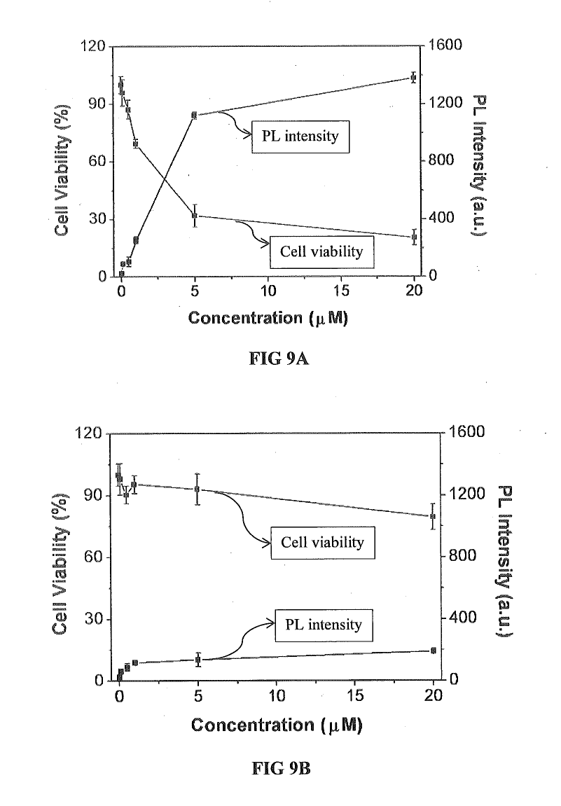

We next compared the relationship between the apoptosis induced fluorescence intensity change in cells and the cytotoxicity profile of the prodrug using both U87-MG and MCF-7 cells as an example. The fluorescence intensities at 480 nm are monitored upon excitation at 365 nm after incubation the cells for 6 h. The cytotoxicity of the cells was evaluated by a standard MTT method after the incubation for 72 h. In this experiment, we studied this effect using both U87-MG and MCF-7 cells. As shown in FIGS. 9A-9B, the cytotoxicity of the prodrug is much more obvious for U87-MG cells which should be due to the higher αᵥβ₃ integrin expression on its surface. The apoptosis sensor TPS-DEVD-NH₂ showed no significant cytotoxicity to both cells. Also, we can find that higher cell viability of the cells will show lower fluorescence intensity which means low degrees of cell apoptosis. For example, when both cells are treated with 5 μM TPS-DEVD-Pt-cRGD, the cell viability is only 31% for U87-MG cells after 72 h incubation, while in sharp contrast, the cell viability is 92% for MCF-7 cells at the same conditions. Meanwhile, in regard to the fluorescence study, the U87-MG cells showed a low degree of fluorescence intensity. This result is also in well accordance with the above apoptosis imaging, indicating that our prodrug can really serve as a targeted drug delivery vehicle and for noninvasive early evaluation of its therapeutic response in situ.

### CONCLUSION

In summary, we report the synthesis and biological application of a theranostic Pt(IV) prodrug for targeted drug delivery and early evaluation of its therapeutic response in situ. The prodrug can be reduced to active Pt(II) inside the cells and simultaneously release the cell apoptosis sensor on its axial position. The reduced Pt(II) can induce the apoptosis of the cancer cell and activate the caspase-3. The activated caspase-3 further cleaves DEVD sequence of the apoptosis sensor and triggers the AIE effect of TPS residue, thus enabling early evaluation of its therapeutic response in cells with high signal-to noise ratios. In addition, we found that the fluorescence intensity induced by apoptosis when incubated with our prodrug for 6 h shows a good correlation with that of the cell viability of the cells determined by MTT assay for 72 h, that is, with lower fluorescence intensity will indicate higher cell viability and vice versa. These results indicate that the theranostic drug delivery system with a build-in apoptosis sensor allows one to evaluate the drug therapeutic response quickly, which is essential to guide therapeutic decisions such as whether the treatment works well or the therapeutic regimes should stop.

### Light-Up Probe PyTPE-Pt-D5-cRGD

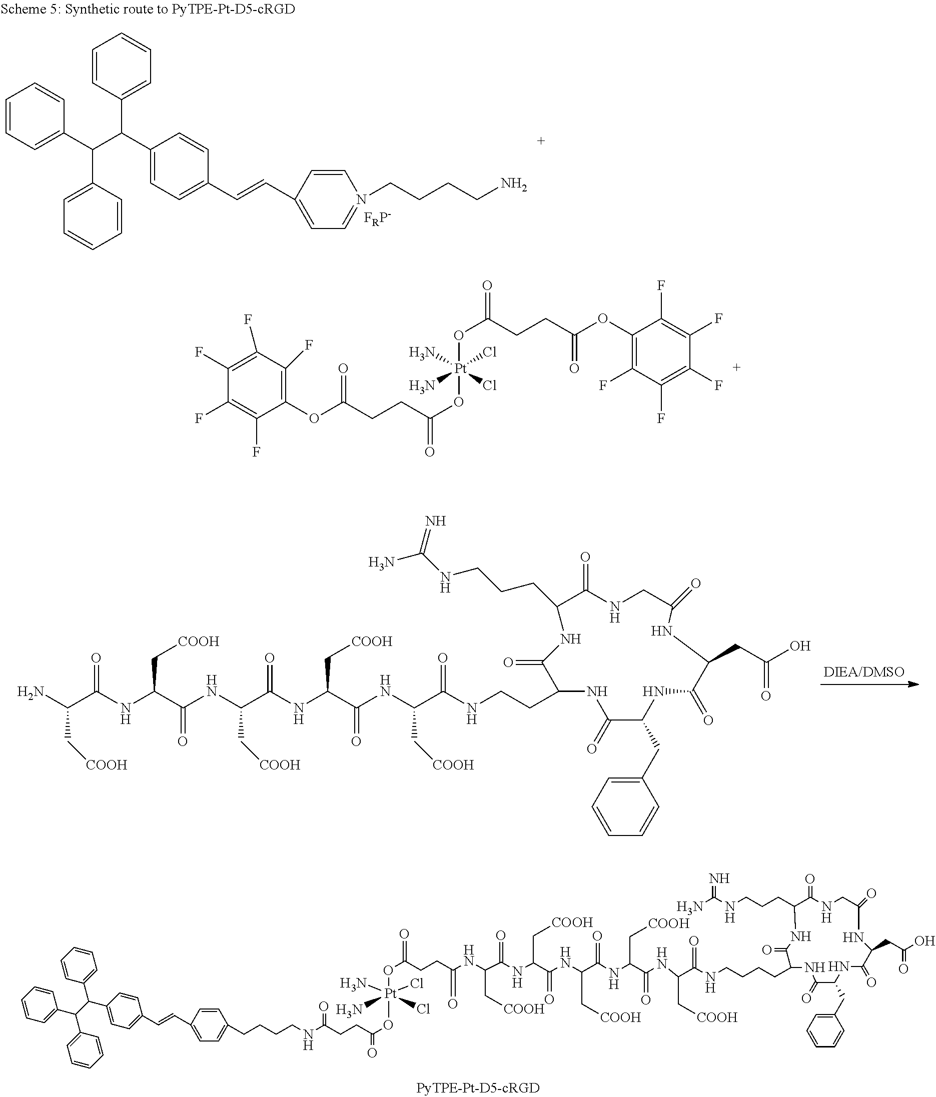

Amine-functionalized PyTPE (PyTPE-NH₂) was synthesized by reducing azide-PyTPE (PyTPE-N₃) in methanol. Pentafluorophenol-activated Pt(IV) complex was prepared from commercially available anticancer drug cisplatin and was used as the linker. The synthetic route for the prodrug PyTPE-Pt-D5-cRGD is shown in Scheme 5. Asymmetric functionalization of activated Pt(IV) complex with PyTPE-NH₂ and amine-functionalized peptide D5-cRGD in the presence of N,N-diisopropylethylamine afforded the prodrug in 42% yield. A control prodrug PyTPE-Pt-D5 with a similar structure but without cRGD moiety was also synthesized in 44% yield. In addition, a non-activatable control PyTPE-C6-D5-cRGD was prepared in 46% yield by using disuccinimidyl suberate to replace activated Pt(IV) complex in the coupling reaction. The NMR and MS characterization confirmed the right structures of the compounds with high purity.

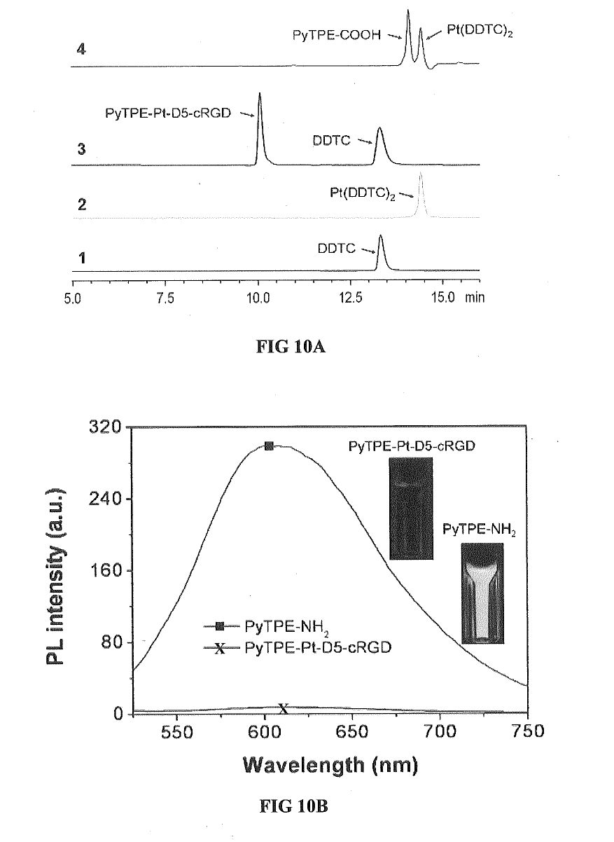

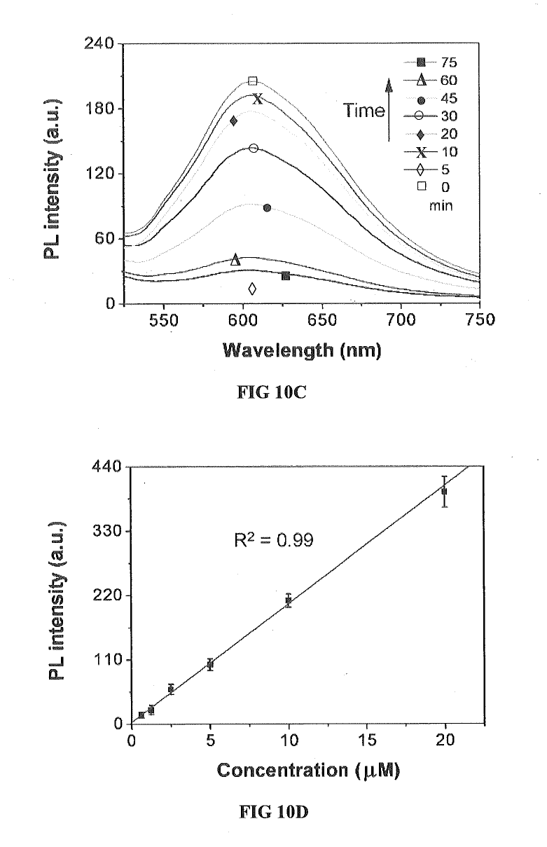

To evaluate our prodrug as a potential anticancer drug, we studied the nature of the formed Pt(II) species upon reduction. It is reported that diethyldithiocarbamate (DDTC) can react with Pt(II) complexes to yield the adducts Pt(DDTC)₂ but does not react with stable Pt(IV) complexes. [10] HPLC-Mass system was used to monitor the adduct formation of Pt(IV) complexes with DDTC after the reduction by ascorbic acid. We choose ascorbic acid as a reduction agent because of its high abundance inside the cells, which has been demonstrated to be a major compound for the reduction of Pt(IV). [11] As shown in FIG. 10A, cisplatin can efficiently bind to DDTC to form the adduct of Pt(DDTC)₂, which shows a different elution time in the HPLC spectrum as compared to free DDTC with a mass-to-charge ratio (m/z) of 492.104. In addition, only in the presence of ascorbic acid, the prodrug can react with DDTC to form Pt(DDTC)_{z}, confirming that the released Pt entities are indeed Pt(II) species. We also find the peak for PyTPE-COOH after reduction, which shows an m/z of 1140.344. Based on these results, we confirm that the prodrug can be reduced by ascorbic acid to generate the reactive platinum(II) drug and release of the axial moieties simultaneously.

The UV-vis absorption spectra of PyTPE-NH₂ in THF and PyTPE-Pt-D5-cRGD in DMSO/PBS (phosphate buffered saline) mixtures (v/v=1/199) were obtained. Both have a similar absorption profile in 348-500 nm with a maximum at 405 nm. The photoluminescence (PL) spectra of PyTPE-NH₂ and PyTPE-Pt-D5-cRGD in DMSO/PBS (v/v=1/199) are shown in FIG. 10B. The hydrophobic PyTPE-NH₂ shows intense fluorescence while PyTPE-Pt-D5-cRGD is almost non-fluorescent in the same mixture, due to easy intramolecular rotations of the TPE phenyl rings in aqueous media. The significant difference in the PL intensities of PyTPE-NH₂ and PyTPE-Pt-D5-cRGD offers opportunity for the prodrug system to be used for real-time monitoring of the drug activation.

To study the response of the prodrug upon reduction, we incubated PyTPE-Pt-D5-cRGD (10 μM) with ascorbic acid (1 mM) in DMSO/PBS (v/v=1/199), and the fluorescence spectra were measured at different time points. As shown in FIG. 10C, the emission intensity of PyTPE-Pt-D5-cRGD increases significantly with time, reaching the plateau in 1.5 h, which is 18-fold higher than its intrinsic emission. The non-targetable prodrug PyTPE-Pt-D5 shows a similar fluorescence intensity increase after the incubation. In contrast, negligible fluorescence intensity increase is observed for PyTPE-C6-D5-cRGD. Further titration of PyTPE-Pt-D5-cRGD with other bioacids and proteins showed negligible fluorescence intensity change, indicating high stability of the pro drug. Only in the presence of the reducing agent (glutathione and ascorbic acid), the prodrug showed an intense fluorescence change. These results indicate that the fluorescence enhancement is due to the reduction of the prodrug.

Next, we incubated different concentrations of prodrug with ascorbic acid (1 mM) and the fluorescence intensities of the prodrug were monitored. The plot of the PL intensities at 605 nm against the pro drug concentration gives a perfect linear line (FIG. 10D), suggesting the possibility of quantification of the drug activation. The gradually enhanced fluorescence intensities are due to the increased amount of PyTPE aggregates formed in aqueous media. The molecular dissolution of the probe and the aggregate formation of the cleaved products were confirmed by laser light scattering (LLS) measurements. In the aqueous mixture, no LLS signals could be detected from the solution of PyTPE-Pt-D5-cRGD. However, after the reduction, the residual hydrophobic AIE luminogen tends to cluster into aggregates with an average size of 145 nm. The non-targetable probe PyTPE-Pt-D5 showed a similar fluorescence intensity increase after incubation with ascorbic acid. Therefore, the drug activation process can be easily monitored on the basis of the PL intensity changes.

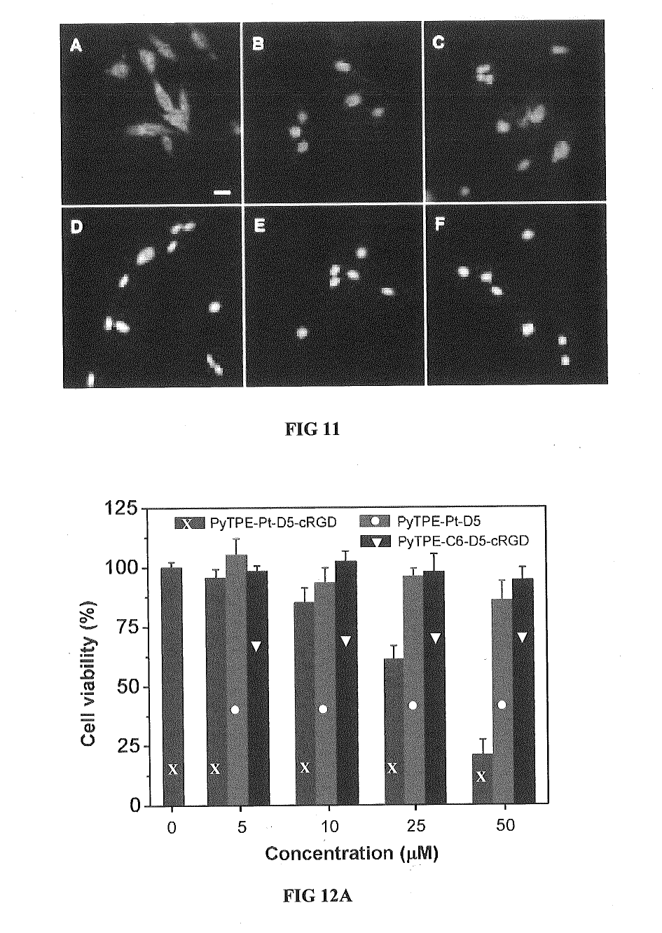

The cell lysates of breast cancer cells MDA-MB-231 were directly incubated with PyTPE-Pt-D5-cRGD (10 μM) and the fluorescence intensity at 605 nm was monitored over time. The fluorescence intensity increases quickly in a similar way to that of the solution study with ascorbic acid in image C of FIG. 11. Meanwhile, the fluorescence intensity shows a minimum change after incubation PyTPE-C6-D5-cRGD with the lysate indicating it is highly stable encounting cellular proteins.

To explore the capability of using PyTPE-Pt-D5-cRGD to monitor targeted intracellular drug reduction in cancer cells, the prodrug was incubated with MDA-MB-231 and MCF-7 breast cancer cell lines. The confocal imaging results are shown in FIG. 11. MDA-MB-231 cells with overexpressed integrin αᵥβ₃ on cellular membrane was chosen as integrin-positive cancer cell, while MCF-7 cells with a low level of integrin αᵥβ₃ expression was used as the negative control. After incubation with PyTPE-Pt-D5-cRGD, the fluorescence in MDA-MB-231 cells is increased gradually with time, whereas for MCF-7 cells only a weak fluorescence signal could be found even after 6 h incubation (image D of FIG. 11). In contrast, PyTPE-Pt-D5 displays weak fluorescence intensity with essentially identical behavior for both cell lines (images B and E of FIG. 11). When MDA-MB-231 cells were pretreated with free cRGD prior to PyTPE-Pt-D5-cRGD incubation, the image showed weak fluorescence. The marked difference reveals that the selective uptake of PyTPE-Pt-D5-cRGD by MDA-MB-231 cells is due to integrin receptor-mediated process. For PyTPE-C6-D5-cRGD, no detectable fluorescence was observed after 6 h's incubation (images C and F of FIG. 11).

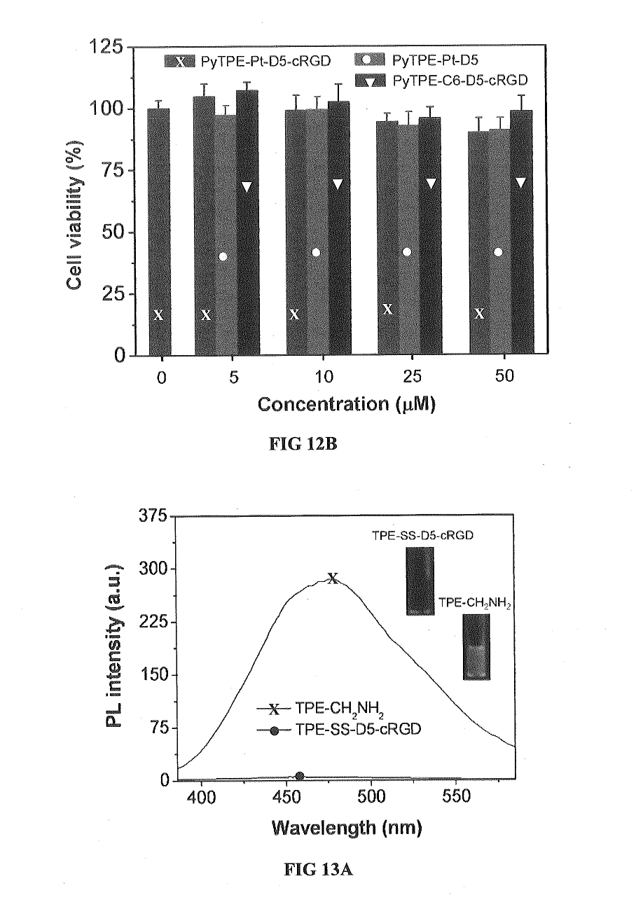

We next studied the cytotoxicity profile of the prodrug to MDA-MB-231 and MCF-7 cells by a MTT assay. As shown in FIGS. 12A-12B, the cytotoxicity of PyTPE-Pt-D5-cRGD is much more obvious for MDA-MB-231 cells which should be due to its higher α_{w}β₃ integrin expression. However, in a parallel experiment for MCF-7 cells, it showed a minimum toxicity. In addition, PyTPE-Pt-D5 and PyTPE-C6-D5-cRGD do not show significant cytotoxicity to both cells. From these results, it is clear that the target moiety in PyTPE-Pt-DS-cRGD plays as a targeting unit to tumor cells and can be reduced to toxic Pt(II) species.

In conclusion, we report the synthesis and biological applications of a fluorescent light-up prodrug based on an AIE luminogen for real-time monitoring of drug activation inside the cells. Thanks to the unique nature of the AIE luminogen, the prodrug is non-fluorescent in aqueous media but becomes highly emissive when reduced inside the cells. The cRGD functionalized peptide allows for selective targeting of αᵥβ₃ integrin on many angiogenic cancers using MDA-MB-231 as an example, which opens new opportunity for specific drug delivery. The prodrug design thus opens new avenues for specific tumor targeting and which permits the concentration of activated drug to be monitored by fluorescence signaling changes.

### Light-Up Probe TPE-SS-D5-cRGD

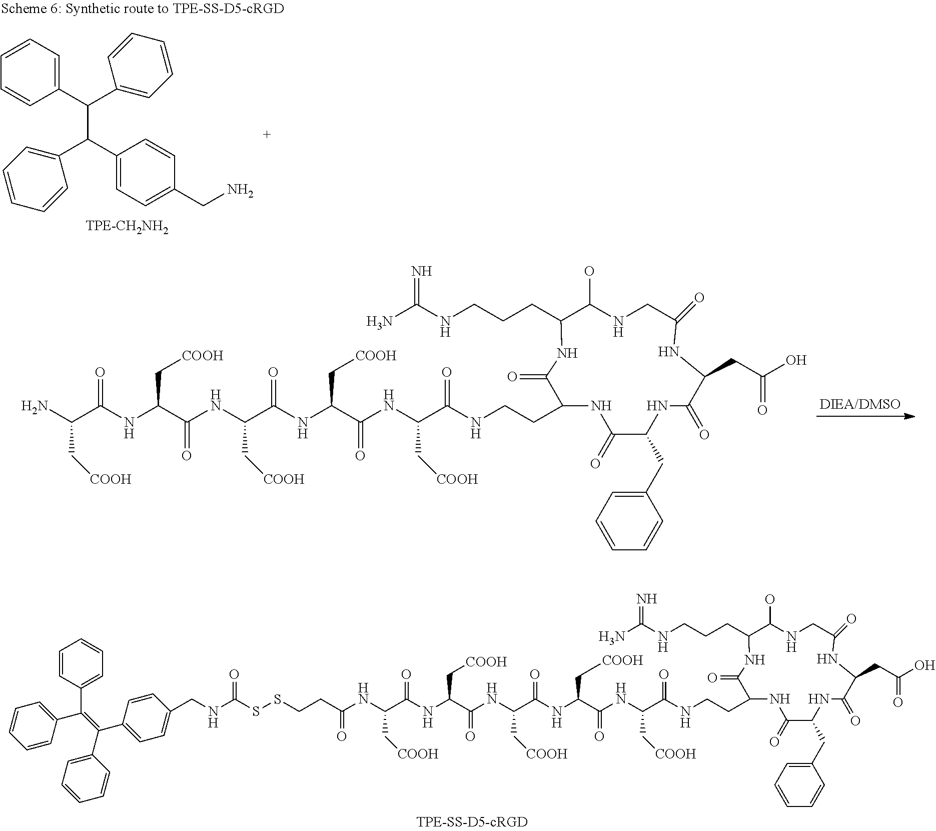 [SMILES: NCc1ccc(C(c2ccccc2)C(c2ccccc2)c2ccccc2)cc1 ; N=C(N)NCCCC1NC(=O)C(CCNC(=O)[C@H](CC(=O)O)NC(=O)[C@H](CC(=O)O)NC(=O)[C@H](CC(=O)O)NC(=O)[C@H](CC(=O)O)NC(=O)[C@@H](N)CC(=O)O)NC(=O)[C@@H](Cc2ccccc2)NC(=O)[C@H](CC(=O)O)NC(=O)CNC1O ; N=C(N)NCCCC1NC(=O)C(CCNC(=O)[C@H](CC(=O)O)NC(=O)[C@H](CC(=O)O)NC(=O)[C@H](CC(=O)O)NC(=O)[C@H](CC(=O)O)NC(=O)[C@H](CC(=O)O)NC(=O)CCSSC(=O)NCc2ccc(C(=C(c3ccccc3)c3ccccc3)c3ccccc3)cc2)NC(=O)[C@@H](Cc2ccccc2)NC(=O)[C@H](CC(=O)O)NC(=O)CNC1O]

Amine-functionalized TPE (TPE-CH₂NH₂) was synthesized by reducing azide-TPE (TPE-CH₂N₃) in methanol. Asymmetric functionalization of DSP linker with TPE-CH₂NH₂ and NH₂ terminated D5-cRGD in the presence of N,N-diisopropylethylamine (DIEA) in anhydrous dimethyl sulfoxide (DMSO) afforded the probe TPE-SS-D5-cRGD in 45% yield (Scheme 6). A control probe TPE-SS-D5 with a similar structure but without cRGD moiety was also synthesized in 49% yield. In addition, a non-activatable control probe TPE-CC-D5 was prepared in 44% yield by using disuccinimidyl suberate to replace DSP in the coupling reaction. The NMR and MS characterizations confirmed the right structures with high purity of the three probes.

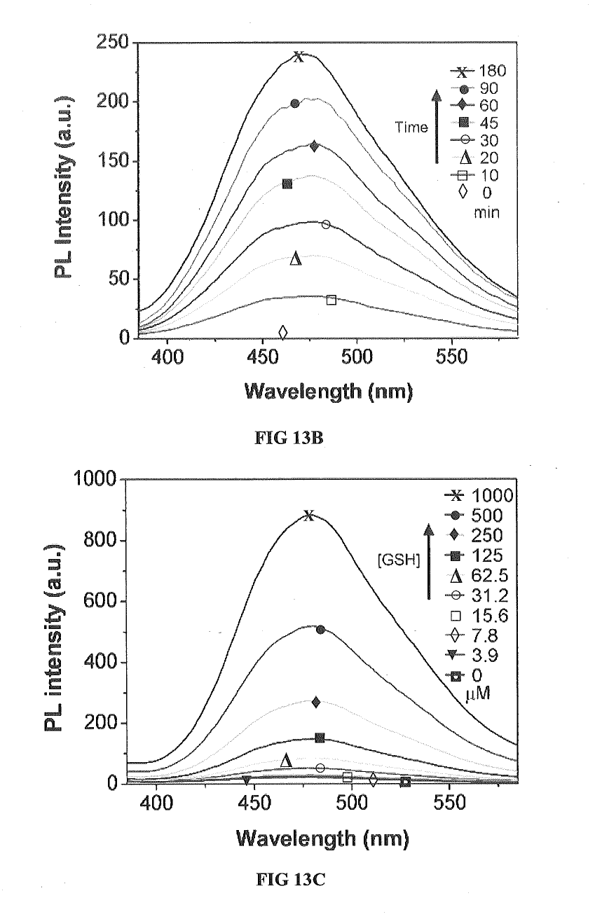

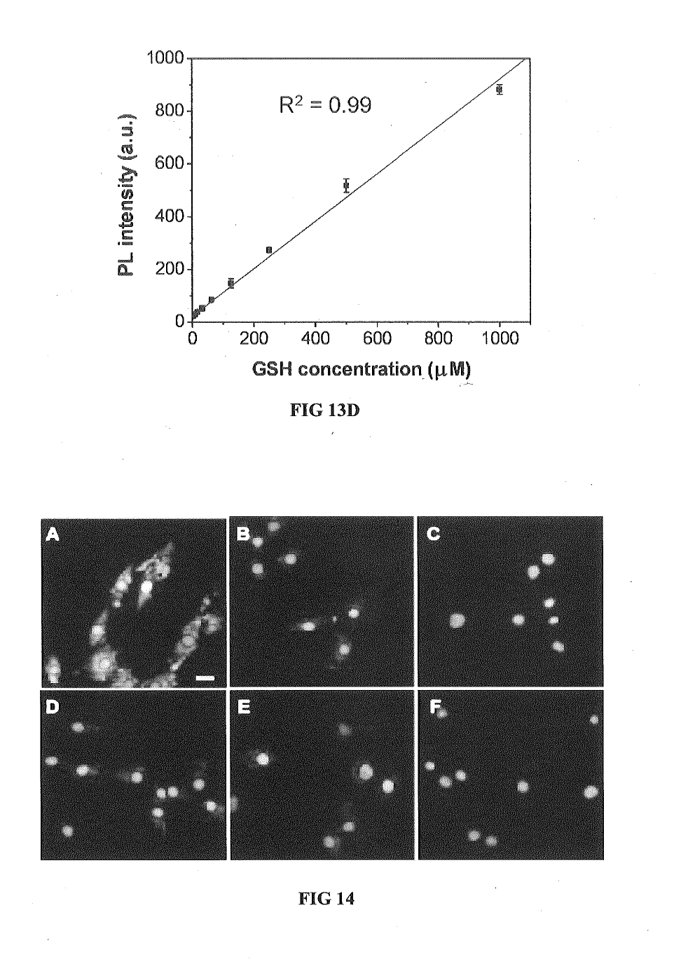

The photoluminescence (PL) spectra of TPE-CH₂NH₂ and TPE-SS-D5-cRGD in DMSO and phosphate buffered saline (PBS, pH=7.4) mixtures (v/v=1/199) are shown in FIG. 13A. The hydrophobic TPE-CH₂NH₂ shows intense fluorescence as nanoaggregates with a quantum yield (Φ) of 0.23±0.01 by using quinoline sulfate as the standard. [12] The TPE-SS-D5-cRGD probe is almost non-fluorescent in the same medium (Φ=0.001), due to the easy intramolecular rotations of the TPE phenyl rings in aqueous media. The significant difference in the PL intensities of TPE-CH₂NH₂ and TPE-SS-D5-cRGD offers opportunity for the probe to be used for specific light-up imaging of thiols. The PL spectrum of TPE-SS-D5-cRGD shows no response to NaCl in the concentration range of 0 to 960 mM. Its PL profile also does not change in the presence of the commonly used cell culture medium Dulbecco's Modified Eagle Medium (DMEM). The probe maintains an “off” state in the complex environment and thus has great potentials to serve as a specific light-up probe with minimum background interference.

To study the response of the probe to free thiols, GSH was chosen as the representative thiol due to its high concentration in the human cellular system. [13] GSH (1 mM) was used to incubate with 10 μM TPE-SS-D5-cRGD in DMSO/PBS mixtures (v/v=1/199), and the fluorescence spectra were measured at different time points. As shown in FIG. 13B, the emission intensity of TPE-SS-D5-cRGD increases significantly with time, reaching the maximum within 3 h, which is 68-fold higher than the intrinsic emission of the probe. The TPE-SS-D5 shows a similar time dependent fluorescence increase after incubation with GSH, while negligible signal is observed for TPE-CC-D5. TPE-SS-D5-cRGD is further demonstrated to response to GSH under acidic conditions.

Next, we investigated the effect of GSH concentration on the probe emission. Different concentrations of GSH ranging from 3.9 μM to 1.0 mM were incubated with TPE-SS-D5-cRGD for 3 h, and the corresponding spectra are shown in FIG. 13C. With increasing GSH concentration, the fluorescence is gradually intensified due to the increased amount of TPE aggregates formed in aqueous media. The molecular dissolution of the probe and the aggregate formation of the cleaved products were confirmed by laser light scattering (LLS) measurements. In the aqueous mixture, no LLS signals could be detected from the solution of TPE-SS-D5-cRGD. However, after incubation with GSH, the residual hydrophobic AIE luminogen tends to cluster into aggregates. The formation of aggregates was further confirmed by AFM. Under the same experimental conditions, a similar fluorescence intensity increase is observed for TPE-SS-D5, but not for TPE-CC-D5. In addition, plotting the PL intensities at 470 nm for TPE-SS-D5-cRGD against the GSH concentration gives a perfect linear line (FIG. 13D), suggesting the possibility of using the probe for GSH quantification with a detection limit of 1.0 μM.

To monitor the GSH-induced fluorescence activation of TPE-SS-D5-cRGD, reverse-phase HPLC and MS analyses were used to follow the exposure of the probe to GSH. After incubation of TPE-SS-D5-cRGD with GSH for 3 h, the mixture was subjected to HPLC analysis. In addition to the TPE-SS-D5-cRGD peak eluted at 10.83 min, two new peaks at 10.68 min for GSS-TPE and 11.58 min for TPE-SH are observed and the peaks show mass-to-charge ratios (m/z) of 755.217 and 472.164 analyzed by IT-TOF, respectively. The fragments of TPE-SH and GSS-TPE tend to aggregate in DMSO/PBS (v/v=1/199), which show blue fluorescence with quantum yields of 19±1% and 12±1%, respectively, using quinoline sulfate as reference. These results clearly demonstrate that the observed GSH-induced fluorescence intensity change of TPE-SS-D5-cRGD is due to cleavage of the disulfide bond, which leads to solubility difference between the probe and the fragment. Further titration of TPE-SS-D5-cRGD with cysteine (Cys), glycine (Gly) and glutamate (Giu), the three amino acids contained in GSH, reveals that the fluorescence turn-on is due to the interaction of free thiol in Cys with the disulfide bond.

To explore the capability of using TPE-SS-D5-cRGD as a specific bioprobe for monitoring intracellular thiol levels in cancer cells, the probe is incubated with U87-MG human glioblastoma and MCF-7 breast cancer cell lines. The confocal imaging results are shown in FIG. 14. U87-MG cells with overexpressed integrin αᵥβ₃ on cellular membrane was chosen as integrin-positive cancer cell, while breast cancer cell MCF-7 with a low level of integrin αᵥβ₃ expression was used as the negative control. After incubation with TPE-SS-D5-cRGD, a strong blue fluorescence is observed for U87-MG cells (image A of FIG. 14), whereas for MCF-7 cells only a weak fluorescence signal could be found even after 6 h incubation (image D of FIG. 14). In contrast, TPE-SS-D5 displays weak fluorescence intensity with essentially identical behavior for both cell lines (images B and E of FIG. 14). When U87-MG cells were pretreated with free cRGD prior to TPE-SS-D5-cRGD incubation, the image showed weak fluorescence. The marked difference reveals that the selective uptake of TPE-SS-D5-cRGD by U87-MG cells is due to integrin receptor-mediated process. For the control probe TPE-CC-D5, no detectable fluorescence was observed even after 6 h incubation (images C and F of FIG. 14). It should be noted that the probe could also be used for live cell imaging.

To provide further evidence for thiol-induced disulfide bond cleavage as the trigger of fluorescence turn-on, the U87-MG cells were also pretreated with buthionine sulfoximine (BSO) before incubation with TPE-SS-D5-cRGD. BSO is an inhibitor of g-glutamylcysteine synthetase which can inhibit the cells from synthesizing GSH. [14] The fluorescence of TPE-SS-D5-cRGD treated U87-MG cells decreases as the concentration of BSO increases from 25 to 100 μM. The significantly reduced fluorescence as compared to that in image A of FIG. 14 reveals that the probe fluorescence is directly related to GSH concentration in the cells. These results indicate that despite the existence of other free thiols in cells, TPE-SS-D5-cRGD could be used as an indicator for intracellular GSH imaging. In vitro cytotoxicity studies also show that the TPE-SS-D5-cRGD probe is biocompatible.

In conclusion, we report the synthesis and biological applications of a light-up GSH responsive AIE probe. Thanks to the unique nature of the AIE luminogen, the probe is nonfluorescent in aqueous media but becomes highly emissive when cleaved by thiols. The probe enables light-up monitoring of free thiols in solution and in cells with a high signal-to-noise ratio. The cRGD functionalized peptide allows for selective targeting of αᵥβ₃ integrin of many angiogenic cancers using U87-MG as an example, which opens new opportunity for specific intracellular thiol imaging. Our AIE probe strategy can be generalized to perform various tasks by simply changing the disulfide groups with other cleavable linkers in chemical biology.

### Light-Up Probe DDT-Gd

The synthetic route to TPE-DEVD-DOTA/Gd (DDT-Gd) is as follows:

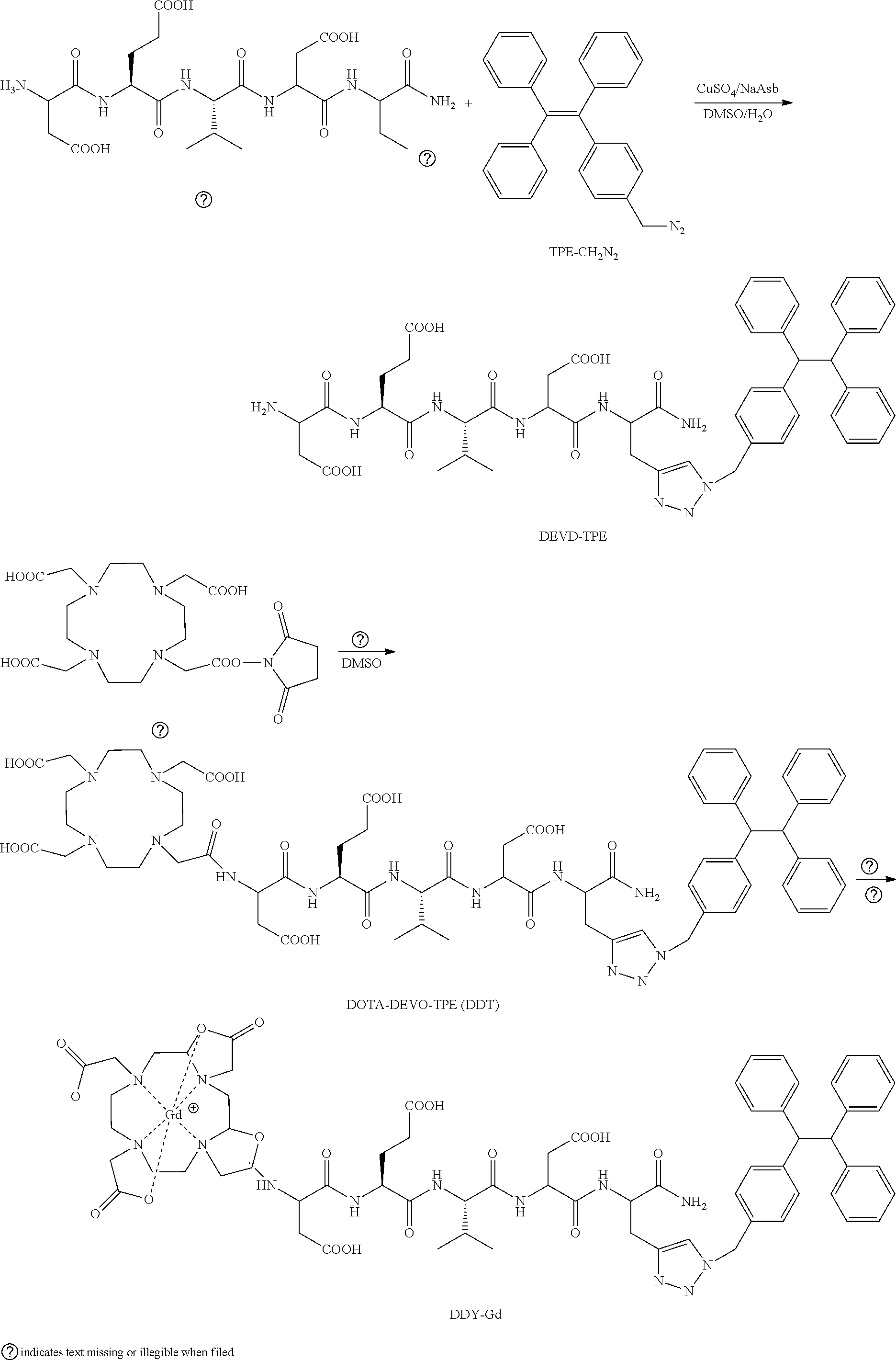

### Synthesis of DEVD-TPE

Excess amount of alkyne bearing peptide (DEVD-alkyne, 22 mg, 38.7 μmol) and azide-functionalized TPE (TPE-CH₂N₃, 10 mg, 25.8 μmol) are dissolved in 0.8 mL of DMSO and vortexed to obtain a clear solution. CuSO₄ (0.5 mg, 9.6 μmol) and sodium ascorbate (2.5 mg, 38.7 μmol) dissolved in 0.2 mL of Milli-Q water were subsequently added into the mixture to initiate click chemistry. The reaction was allowed to proceed at 37° C. under shaking for ˜2 days. The product TPE-DEVD was then purified by HPLC with a yield of 60% and further characterized by LC-MS and NMR.

### Synthesis of DOTA-DEVD-TPE (DDT)

The as-synthesized DEVD-TPE (10 mg, 10.4 μmol) and excess amount of DOTA-NHS ester (10.4 mg, 20.8 mop were dissolved in a total of 0.6 mL of DMSO and mixed thoroughly by vortexing. The reaction was allowed to proceed at room temperature for ˜2 days under shaking. The product DDT was then purified by HPLC with a yield of 70% and further characterized by LC-MS and NMR. IT-TOF-MS: m/z [M+2H]²⁺ calc. 672.799. found 672.782.

### Synthesis of DDT-Gd

The as-synthesized DDT product (10 mg, 7.4 μmol) was dissolved in 0.4 mL of DMSO. GdCl₃ (9.8 mg, 37 mop was dissolved in 0.4 mL of Milli-Q water with pH adjusted to 5 using NaOH. GdCl₃ solution was then added into DDT and the mixture was mixed thoroughly by vortexing. The reaction mixture was shaken at room temperature to further react for ˜4 days. The product DDT-Gd was then purified by HPLC with a yield of 60% and further characterized by LC-MS and NMR. IT-TOF-MS: m/z [M+2H]²⁺ calc. 750.249. found 750.222.

### REFERENCES

[1] K. R. Barnes, A. Kutikov, S. J. Lippard, Chemistry & Biology 2004, 11, 557.
[2] J. Z. Liu, R. H. Zheng, Y. H. Tang, M. Haussler, J. W. Y. Lam, A. Qin, M. X. Ye, Y. N. Hong, P. Gao, B. Z. Tang, Macromolecules 2007, 40, 7473.
[3] A. Lopez-Flores, R. Jurado, P. Garcia-Lopez, J Pharmacal Toxicol Methods 2005, 52, 366.
[4] J. Li, S. Q. Yap, C. F. Chin, Q. Tian, S. L. Yoong, G. Pastorin, W. H. Ang, Chemical Science 2012, 3, 2083.
[5] N. Graf, S. J. Lippard, Advanced Drug Delivery Reviews 2012, 64, 993.
[6] K. L. D. Ding, B. Liu, and B. Z. Tang, Acc. Chem. Res. 2013, DOl: 10.1021/ar3003464.
[7] Y. N. Hong, J. W. Y. Lam, B. Z. Tang, Chemical Society Reviews 2011, 40, 5361.
[8] D. Lee, S. A. Long, J. L. Adams, G. Chan, K. S. Vaidya, T. A. Francis, K. Kikly, J. D. Winkler, C. M. Sung, C. Debouck, S. Richardson, M. A. Levy, W. E. DeWolf, P. M. Keller, T. Tomaszek, M. S. Head, M. D. Ryan, R. C. Haltiwanger, P. H. Liang, C. A. Janson, P. J. McDevitt, K. Johanson, N. 0. Concha, W. Chan, S. S. Abdel-Meguid, A. M. Badger, M. W. Lark, D. P. Nadeau, L. J. Suva, M. Gowen, M. E. Nuttall, Journal of Biological Chemistry 2000, 275, 16007.
[9] A. Luhrmann, C. V. Nogueira, K. L. Carey, C. R. Roy, Proceedings of the National Academy of Sciences of the United States of America 2010, 107, 18997.
[10] J. Li, S. Q. Yap, C. F. Chin, Q. Tian, S. L. Yoong, G. Pastorin and W. H. Ang, Chemical Science, 2012, 3, 2083-2087.
[11] N. Graf and S. J. Lippard, Advanced Drug Delivery Reviews, 2012, 64, 993-1004.
[12] Demas, J. N.; Crosby, G. A. J Phys. Chem. 1971, 75, 991.
[13] Hwang, C.; Sinskey, A. J.; Lodish, H. F. Science 1992, 257, 1496.
[14] Hultberg, M.; Hultberg, B. Chem. Biol. Interact. 2006, 163, 192.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A chemical composition, comprising: a target recognition motif, a hydrophilic moiety, a linking moiety, and at least one luminogen, wherein the luminogen exhibits aggregation-induced emission properties, and further wherein the target recognition motif, the hydrophilic moiety, the linking moiety, and the at least one luminogen are linked by covalent linkages in a linear array.

2. The composition of claim 1, wherein the linking moiety is a prodrug or a cleavable linking group.

3. The composition of claim 2, wherein the prodrug is a platinum (IV) complex.

4.-5. (canceled)

6. The composition of claim 1, wherein the luminogen is tetraphenylethylene, tetraphenylsilole, or a luminogen having the structure of formula: 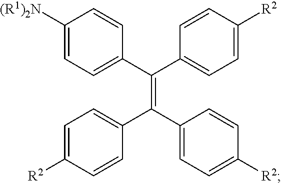
or a pharmaceutically acceptable salt thereof, wherein:
R¹ is selected from H, (C₁-C₆)alkyl, (C₃-C₆)cycloalkyl, (C₆-C₁₀)aryl, (C₃-C₁₀)heteroaryl, or (C₂-C₆)alkenyl;
each R² is independently selected from H, NHR³, N(R³)₂, (C₁-C₆)alkyl, (C₃-C₆)cycloalkyl, (C₆-C₁₀)aryl, (C₃-C₁₀)heteroaryl, —O(C₁-C₆)alkyl, (C₂-C₆)alkenyl, CH═CH((C₃-C₁₀)heteroaryl), or CH═CH((C₆-C₁₀)aryl); and
R³ is selected from H, (C₁-C₆)alkyl or (C₃-C₆)cycloalkyl;
and wherein the luminogen is optionally and independently substituted with one or more substituents selected from:
(C₃-C₁₀)heteroaryl, 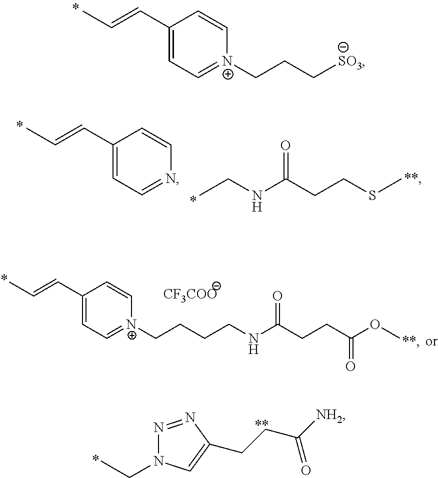
wherein * indicates the point of attachment to the luminogen residue and ** indicates the point of attachment to either the prodrug, the target recognition motif or the hydrophilic peptide.

7. The composition of claim 1, wherein the luminogen has the structure of formula: 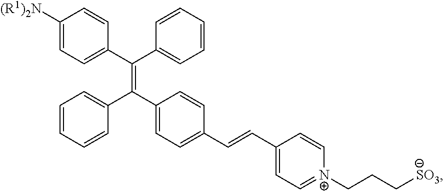
wherein R¹ is C₂H₅ or C₆H₁₃; or wherein the luminogen has the structure of formula:

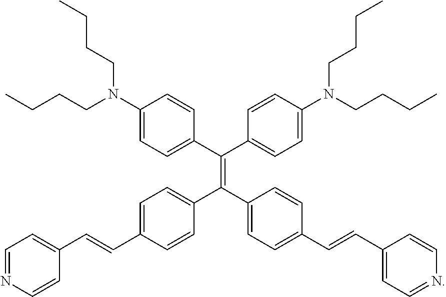 [SMILES: CCCCN(CCCC)c1ccc(C(=C(c2ccc(/C=C/c3ccncc3)cc2)c2ccc(/C=C/c3ccncc3)cc2)c2ccc(N(CCCC)CCCC)cc2)cc1]

8. The composition of claim 1, wherein the hydrophilic moiety comprises a hydrophilic peptide, a self-assembling peptide, an oligonucleotide, a water soluble polymer, or an alkyl chain functionalized by charged side groups.

9.-10. (canceled)

11. The composition of claim 8, wherein the self-assembling peptide is (Ala-Glu-Ala-Glu-Ala-Lys-Ala-Lys)₂ (SEQ ID NO:3).

12. The composition of claim 1, wherein the target recognition motif has an affinity for a cell membrane receptor or a cyclic(Arg-Gly-Asp) residue having an affinity for integrin αᵥβ₃.

13.-14. (canceled)

15. The composition of claim 2, wherein the target recognition motif is covalently bonded to the hydrophilic peptide, the hydrophilic peptide is covalently bonded to the prodrug, and the prodrug is covalently bonded to the luminogen.

16. The composition of claim 2, wherein the target recognition motif is covalently bonded to the prodrug, the prodrug is covalently bonded to the hydrophilic peptide, and the hydrophilic peptide is covalently bonded to the luminogen.

17. The composition of claim 2, wherein the target recognition motif is covalently bonded to the cleavable linking group, the cleavable linking group is covalently bonded to the hydrophilic peptide, and the hydrophilic peptide is covalently bonded to the luminogen.

18. The composition of claim 1, having the structure of formula: 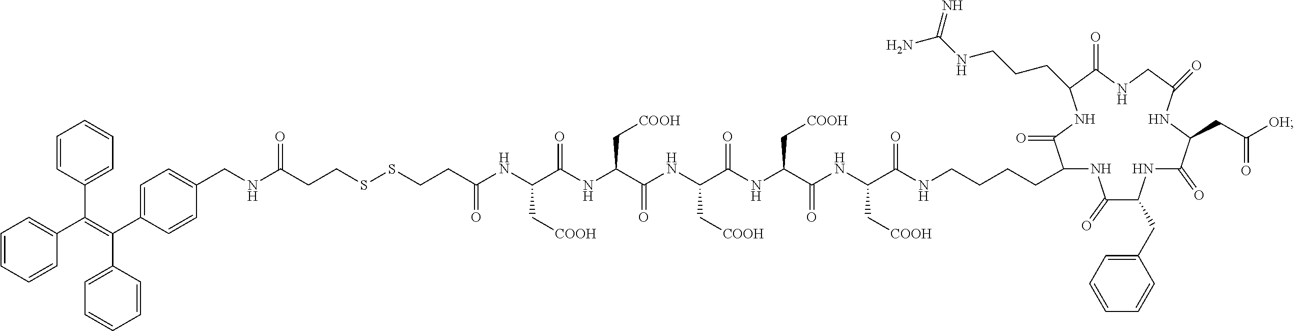 [SMILES: N=C(N)NCCCC1NC(=O)C(CCCCNC(=O)[C@H](CC(=O)O)NC(=O)[C@H](CC(=O)O)NC(=O)[C@H](CC(=O)O)NC(=O)[C@H](CC(=O)O)NC(=O)[C@H](CC(=O)O)NC(=O)CCSSCCC(=O)NCc2ccc(C(=C(c3ccccc3)c3ccccc3)c3ccccc3)cc2)NC(=O)[C@@H](Cc2ccccc2)NC(=O)[C@H](CC(=O)O)NC(=O)CNC1=O]
or a pharmaceutically acceptable salt thereof.

19. The composition of claim 15, having the structure of formula: 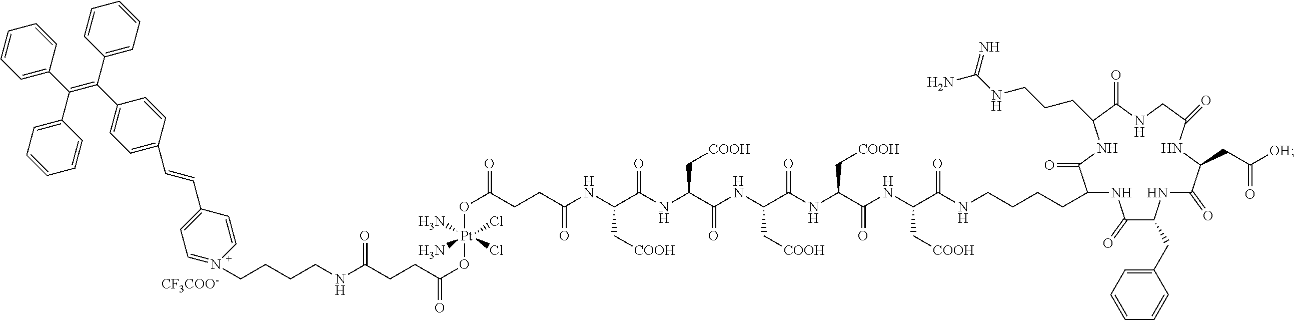
or a pharmaceutically acceptable salt thereof.

20. The composition of claim 16, having the structure of formula: 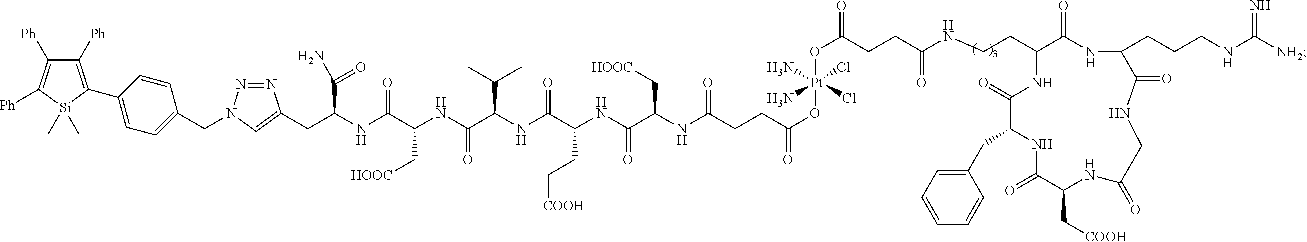
or a pharmaceutically acceptable salt thereof.

21. A method for assessing the conversion of a prodrug into its active form, comprising:
a) incubating a biological sample with the composition of claim 15 under conditions sufficient to form an incubated mixture; and
b) analyzing the fluorescence of the incubated mixture of step a) using a microplate reader, wherein an increase in fluorescence intensity as compared to the fluorescence intensity of the composition of claim 15 not in the presence of the biological sample is indicative of the conversion of the prodrug into its active form.

22. (canceled)

23. A method for assessing the therapeutic efficacy of a prodrug, comprising:
a) incubating a biological sample comprising live target cells with the composition of claim 16 under conditions sufficient to convert the prodrug into its active form and form an incubated mixture; and
b) analyzing the incubated mixture of step a) by fluorescence spectroscopy, wherein an increase in fluorescence intensity as compared to the fluorescence intensity of the composition of claim 16 not in the presence of the biological sample is indicative of the efficacy of the active drug.

24. A method for detecting glutathione in a biological sample, comprising:
a) incubating a biological sample thought to contain glutathione with the composition of claim 1 under conditions sufficient to form an incubated mixture; and
b) analyzing the incubated mixture of step a) by fluorescence spectroscopy, wherein an increase in fluorescence intensity as compared to the fluorescence intensity of the composition of claim 1 not in the presence of the biological sample is indicative presence of glutathione.

25. (canceled)

26. A method for the detection of alkaline phosphatase in a sample, comprising:
a) incubating a sample thought to comprise alkaline phosphatase with a compound of the formula: 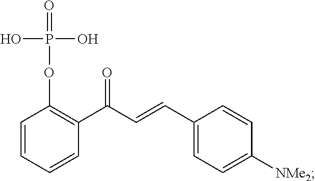 [SMILES: CN(C)c1ccc(/C=C/C(=O)c2ccccc2OP(=O)(O)O)cc1]
under conditions sufficient to form an incubated media; and
b) analyzing the incubated media of step a) by fluorescence spectroscopy, wherein an increase in fluorescence intensity of a fluorescence signal at about 641 nm is indicative of the presence of alkaline phosphatase.

27. (canceled)

28. A chemical composition, comprising a compound of the formula: 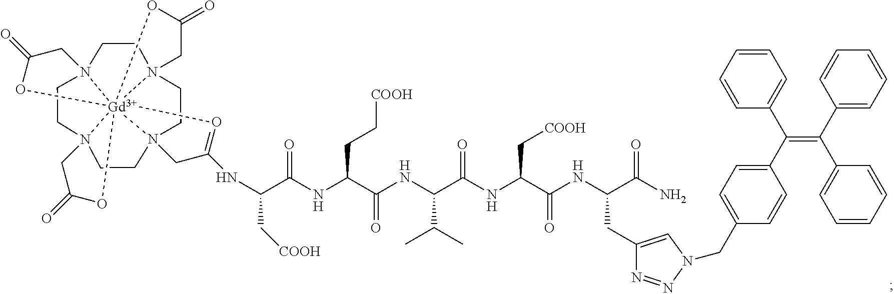
or a pharmaceutically acceptable salt thereof.

29. A method comprising conducting fluorescence imaging or magnetic resonance imaging wherein said conducting of said fluorescence imaging or said magnetic resonance imaging utilizes the composition of claim 28.

30. A luminogen having the structure of formula: 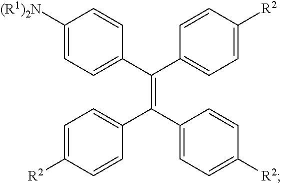
or a pharmaceutically acceptable salt thereof, wherein:
R¹ is selected from H, (C₁-C₆)alkyl, (C₃-C₆)cycloalkyl, (C₆-C₁₀)aryl, (C₃-C₁₀)heteroaryl, or (C₂-C₆)alkenyl;
each R² is independently selected from H, NHR³, N(R³)₂, (C₁-C₆)alkyl, (C₃-C₆)cycloalkyl, (C₆-C₁₀)aryl, (C₃-C₁₀)heteroaryl, —O(C₁-C₆)alkyl, (C₂-C₆)alkenyl, CH═CH((C₃-C₁₀)heteroaryl), or CH═CH((C₆-C₁₀)aryl); and
R³ is selected from H, (C₁-C₆)alkyl or (C₃-C₆)cycloalkyl.

31. The luminogen of claim 30, having the structure of formula: 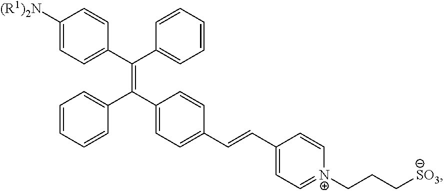
wherein:
R¹ is (C₁-C₆)alkyl.

32. The luminogen of claim 31, wherein R¹ is C₂H₅ or C₆H₁₃.

33. The luminogen of claim 30, having the structure of formula:

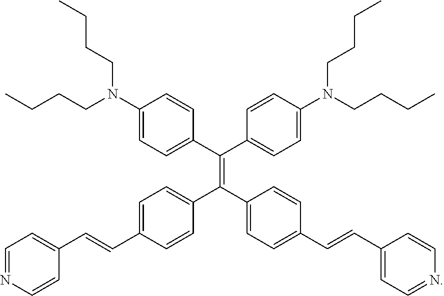 [SMILES: CCCCN(CCCC)c1ccc(C(=C(c2ccc(/C=C/c3ccncc3)cc2)c2ccc(/C=C/c3ccncc3)cc2)c2ccc(N(CCCC)CCCC)cc2)cc1]

34. A luminogen having the structure of formula: 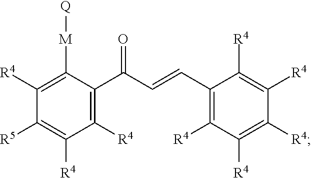
or a pharmaceutically acceptable salt thereof, wherein:
M is selected from S, O or NH;
Q is selected from P(═O)(OH)₂ or C(O)O(C₁-C₆)alkyl;
wherein C(O)O(C₁-C₆)alkyl is optionally functionalized by one or more substituents selected from SH, OH, NH₂, or (C₆-C₁₀)aryl optionally substituted with one or more substituents selected from OH, SH, or NH₂;
R⁴ is selected from NHR⁶, N(R⁶)₂, (C₁-C₆)alkyl, (C₃-C₆)cycloalkyl, (C₆-C₁₀)aryl, (C₃-C₁₀)heteroaryl, —O(C₁-C₆)alkyl, —O(C₃-C₆)cycloalkyl or (C₂-C₆)alkenyl;
R⁵ is (C₀-C₆)alkyl, optionally functionalized by a linking moiety; and
R⁶ is selected from H, (C₁-C₆)alkyl, or (C₃-C₆)cycloalkyl.

35. (canceled)


SEQUENCE LISTING (3 sequences)

SEQ ID NO: 1 - protein, 5 aa, Artificial Sequence
  Artificially Synthesized Protein
DDDDD

SEQ ID NO: 2 - protein, 4 aa, Artificial Sequence
  Artificially Synthesized Protein
DEVD

SEQ ID NO: 3 - protein, 16 aa, Artificial Sequence
  Artificially Synthesized Protein
AEAEAKAKAEAEAKAK